SEQUENCE LISTING (13 sequences)

SEQ ID NO: 1 - DNA, 29 bp, synthetic construct
  source 1..29: mol_type = other DNA
  modified_base 23: mod_base = OTHER; note = uracil
aatgatacggcgaccaccgagatctacac

SEQ ID NO: 2 - DNA, 29 bp, synthetic construct
  source 1..29: mol_type = other DNA
  modified_base 23: mod_base = i
aatgatacggcgaccaccgaganctacac

SEQ ID NO: 3 - DNA, 29 bp, synthetic construct
  source 1..29: mol_type = other DNA
  modified_base 23: mod_base = OTHER; note = alkene-thymidine
aatgatacggcgaccaccgaganctacac

SEQ ID NO: 4 - DNA, 24 bp, synthetic construct
  source 1..24: mol_type = other DNA
  modified_base 22: mod_base = OTHER; note = 8-oxoguanine
caagcagaagacggcatacganat

SEQ ID NO: 5 - DNA, 24 bp, synthetic construct
  source 1..24: mol_type = other DNA
  modified_base 20: mod_base = OTHER; note = 8-oxoguanine
caagcagaagacggcatacnagat

SEQ ID NO: 6 - DNA, 24 bp, synthetic construct
  source 1..24: mol_type = other DNA
  modified_base 20: mod_base = OTHER; mod_base = OTHER; note = 8-oxoguanine
  modified_base 23: mod_base = OTHER; note = 8-oxoguanine
caagcagaagacggcatacnanat

SEQ ID NO: 7 - DNA, 24 bp, synthetic construct
  source 1..24: mol_type = other DNA
  modified_base 22: mod_base = OTHER; note = uracil
caagcagaagacggcatacgatat

SEQ ID NO: 8 - DNA, 24 bp, synthetic construct
  source 1..24: mol_type = other DNA
  modified_base 20: mod_base = OTHER; note = uracil
caagcagaagacggcatactagat

SEQ ID NO: 9 - DNA, 29 bp, synthetic construct
  source 1..29: mol_type = other DNA
  modified_base 23: mod_base = OTHER; note = allyl-T
aatgatacggcgaccaccgaganctacac

SEQ ID NO: 10 - DNA, 35 bp, synthetic construct
  source 1..35: mol_type = other DNA
gctggcacgtccgaacgcttcgttaatccgttgag

SEQ ID NO: 11 - DNA, 35 bp, synthetic construct
  source 1..35: mol_type = other DNA
cgtcgtctgccatggcgcttcggtggatatgaact

SEQ ID NO: 12 - DNA, 35 bp, synthetic construct
  source 1..35: mol_type = other DNA
acggccgctaatatcaacgcgtcgaatccgcaact

SEQ ID NO: 13 - DNA, 34 bp, synthetic construct
  source 1..34: mol_type = other DNA
gccgcgttacgttagccggactattcgatgcagc

(19) United States
(12) Patent Application Publication
(10) Pub. No.: US 2024/0207839 A1
(43) Pub. Date: Jun. 27, 2024

(54) FLOW CELLS

(71) Applicant: ILLUMINA, INC., San Diego, CA (US)

(72) Inventors: Johan Sebastian Basuki, Singapore (SG); Brian D. Mather, San Diego, CA (US); Sebastien Georg Gabriel Ricoult, Cambridge (GB); Alexandra Szemjonov, Cambridge (GB)

(21) Appl. No.: 18/542,470

(22) Filed: Dec. 15, 2023

Related U.S. Application Data
(60) Provisional application No. 63/586,716, filed on Sep. 29, 2023.
(60) Provisional application No. 63/387,874, filed on Dec. 16, 2022.

(51) Int. Cl. B01L 3/00

(52) U.S. Cl. CPC B01L 3/502; B01L 2300/069; B01L 2300/0816; B01L 2300/0829; B01L 2300/0877; B01L 2300/165

(57) ABSTRACT

An example of a flow cell includes a substrate; a plurality of reactive regions spatially separated from one another across the substrate; and a plurality of independently removable coatings respectively positioned over each of the plurality of reactive regions. Each of the plurality of reactive regions includes a polymeric hydrogel layer; and a reactive entity attached to the polymeric hydrogel layer. At least one of the independently removable coatings is a composite that includes a thermo-responsive polymer and a photo-thermal additive.

### CROSS-REFERENCE TO RELATED APPLICATIONS

This application claims the benefit of U.S. Provisional Application Ser. No. 63/586,716, filed Sep. 29, 2023 and of U.S. Provisional Application Ser. No. 63/387,874, filed Dec. 16, 2022, the contents of each of which are incorporated by reference herein in its entirety.

### REFERENCE TO SEQUENCE LISTING

The Sequence Listing submitted herewith is hereby incorporated by reference in its entirety. The name of the file is ILI251B2_IP-2639-US_Sequence_Listing.xml, the size of the file is 17,866 bytes, and the date of creation of the file is Dec. 10, 2023.

### BACKGROUND

Some biological and/or chemical vessels, such as assay plates and flow cells, include designated reaction areas, where surface chemistry that enables a desired interaction or reaction is localized. When a reactive species is introduced into the vessel, the reactive species interacts or reacts with the surface chemistry to create a detectable signal (e.g., an electrical signal or an optical signal). Many vessels are configured with multiple reaction areas in fluid communication with a single flow channel. In these vessels, a single sample may be introduced into the flow channel and its associated reaction areas, or multiple samples may be pooled and introduced into the flow channel and its associated reaction areas.

### SUMMARY

The biological and/or chemical vessels disclosed herein include a plurality of reactive regions spatially separated from one another across a substrate. These reactive regions include respective reactive entities that may be the same or different.

In some examples disclosed herein, each reactive region is coated with an independently removable coating, which may be a photo-reactive coating. These independently removable coatings enable controlled access to the reactive regions. For example, one or more coating(s) may be removed via light exposure, while one or more other coating(s) remain(s) intact. The reactive region(s) exposed by coating removal become(s) active and thus is/are able to participate in the designated reaction. The reactive region(s) having its/their coating(s) intact remain(s) passivated or protected, and thus remain(s) inactive.

In some other examples disclosed herein, each reactive region is independently electrically addressable. The independent electrical addressability enables a conductive fluid to be directed to a particular reactive region, while other reactive regions remain free of the fluid. Controlled fluid exposure enables the designated reaction to take place at a particular reactive region. Non-exposed reactive regions remain inactive.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a top view of a flow cell;

FIG. 1B is a semi-schematic, partially cross-sectional and partially perspective view of an example architecture of the flow cell of FIG. 1A;

FIG. 1C is a semi-schematic, partially cross-sectional and partially perspective view of another example architecture of the flow cell of FIG. 1A;

FIG. 2A is a schematic cross-sectional view of reactive areas defined in depressions and coated with different removable coatings;

FIG. 2B is a schematic cross-sectional view of reactive areas defined in depressions and coated with different removable coatings, some of which include multiple sub-layers;

FIG. 2C is a schematic cross-sectional view of reactive areas defined in depressions and coated with the same removable coating having different thicknesses;

FIG. 3A is a schematic cross-sectional view of reactive areas defined on protrusions and coated with different removable coatings;

FIG. 3B is a schematic cross-sectional view of reactive areas defined on protrusions and coated with different removable coatings, some of which include multiple sub-layers;

FIG. 3C is a schematic cross-sectional view of reactive areas defined on protrusions and coated with the same removable coating having different thicknesses;

FIG. 3D is a schematic cross-sectional view of reactive areas defined on protrusions and coated with a single removable coating having a step-wise thickness gradient;

FIG. 3E is a schematic cross-sectional view of reactive areas defined on protrusions and coated with a single removable coating having a linear thickness gradient;

FIG. 4 schematically illustrates two example methods (A.-E. or A., B., F., G., E.) using two photo-reactive protective layers, where:

A. illustrates the exposure of one of the photo-reactive protective layers to light, B. illustrates the introduction of a first library template strand, C. illustrates the seeding of the first library template strand and the exposure of the other of the photo-reactive protective layers to light, D. illustrates the introduction of a second library template strand, E. illustrates the amplified template strands; and

A. illustrates the exposure of one of the photo-reactive protective layers to light, B. illustrates the introduction of a first library template strand, F. illustrates the seeding and amplification of the first library template strand and the exposure of the other of the photo-reactive protective layers to light, G. illustrates the introduction of a second library template strand, E. illustrates the amplified template strands;

FIG. 5A is a chemical structure of one example of the photo-reactive protective layer that includes a hydrophilic polymer cross-linked with a photo-cleavable cross-linker;

FIG. 5B depicts a chemical structure of another example of the photo-reactive protective layer that includes a hydrophilic polymer cross-linked with a photo-cleavable cross-linker, and a reaction involving the photo-reactive protective layer;

FIG. 5C depicts a chemical structure of yet another example of the photo-reactive protective layer that includes a hydrophilic polymer cross-linked with a photo-cleavable cross-linker, and another reaction involving the photo-reactive protective layer;

FIG. 6A is a chemical structure of an example of the photo-reactive protective layer that includes a polymer capped with a photo-cleavable hydrophobic group or an acid-labile group;

FIG. 6B depicts a chemical structure of another example of the photo-reactive protective layer (that includes a polymer capped with a photo-cleavable hydrophobic group or an acid-labile group), and a reaction involving the photo-reactive protective layer;

FIG. 7 is a schematic illustration of a digital fluidics system;

FIG. 8A is a top view of a resin layer of a digital fluidics cartridge patterned with three different reactive regions;

FIG. 8B is a top view of an array of individually addressable control electrodes of an instrument;

FIG. 8C is a top view illustrating the resin layer of FIG. 8A overlaid on the array of individually addressable control electrodes of FIG. 8B; and

FIG. 9 is a schematic illustration of a complementary metal-oxide semiconductor imaging device that is coupled to a substrate.

### DEFINITIONS

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

An “acrylamide monomer” is a monomer with the structure

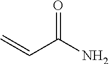

or a monomer including an acrylamide group. Examples of the monomer including an acrylamide group include azido acetamido pentyl acrylamide:

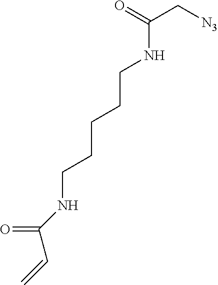 [SMILES: C=CC(=O)NCCCCCNC(=O)CN=[N+]=[N-]]

### and N-isopropylacrylamide:

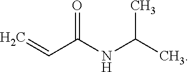

Other acrylamide monomers may be used.

The term “activation,” as used herein, refers to a process that generates reactive groups at the surface of a substrate. Activation may be accomplished using silanization or plasma ashing. While the figures do not depict a separate silanized layer or hydroxyl (—OH groups) from plasma ashing, it is to be understood that activation generates a silanized layer or —OH groups at the surface of the activated support or layer to covalently attach the functionalized layers to the underlying support or layer.

An aldehyde, as used herein, is an organic compound containing a functional group with the structure —CHO, which includes a carbonyl center (i.e., a carbon double-bonded to oxygen) with the carbon atom also bonded to hydrogen and an R group, such as an alkyl or other side chain. The general structure of an aldehyde is:

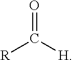

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amine” or “amino” functional group refers to an —NRₐR_{b} group, where Rₐ and R_{b} are each independently selected from hydrogen

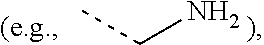

C1-6 (or C1-C6) alkyl, C2-6 alkenyl, C2-6 alkynyl, C3-7 carbocycle, C6-10 aryl, 5-10 membered heteroaryl, and 5-10 membered heterocyclyl, as defined herein.

As used herein, the term “attached” refers to the state of two things being joined, fastened, adhered, connected or bound to each other, either directly or indirectly. For example, a nucleic acid can be attached to a polymeric hydrogel layer by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

As used herein, a “bonding region” refers to an area of a patterned structure that is to be bonded to another material, which may be, as examples, a spacer layer, a lid, another patterned structure, etc., or combinations thereof (e.g., a spacer layer and a lid, or a spacer layer and another patterned structure). The bond that is formed at the bonding region may be a chemical bond, or a mechanical bond (e.g., using a fastener, etc.).

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl.

As used herein, the term “carboxylic acid” or “carboxyl” as used herein refers to —COOH.

As used herein, “cycloalkylene” means a fully saturated carbocycle ring or ring system that is attached to the rest of the molecule via two points of attachment.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocycle ring or ring system with at least one heteroatom in ring backbone, having at least one triple bond, wherein no ring in the ring system is aromatic.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature, defined in a substrate, and having a surface opening that is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” (also referred to as a glycidyl or oxirane group) as used herein refers to

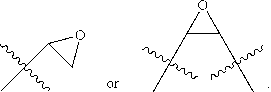

As used herein, the term “flow cell” is intended to mean a vessel having a flow channel where a reaction can be carried out, an inlet for delivering reagent(s) to the flow channel, and an outlet for removing reagent(s) from the flow channel. In some examples, the flow cell accommodates the detection of the reaction that occurs in the flow cell. For example, the flow cell can include one or more transparent surfaces allowing for the optical detection of arrays, optically labeled molecules, or the like.

As used herein, a “flow channel” or “channel” may be an area defined between two bonded components or an area defined in a patterned structure that is open to an external environment, either of which can selectively receive a liquid sample. In some examples, the flow channel may be defined between two patterned structures, and thus may be in fluid communication with surface chemistry of each of the patterned structures. In other examples, the flow channel may be defined between a patterned structure and a lid, and thus may be in fluid communication with surface chemistry of the one patterned structure. In still other examples, the flow channel may be defined in a substrate of a patterned structure such that it is open to an external environment.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen, and/or sulfur in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

As used herein, the term “hydrazone” or “hydrazonyl” refers to a

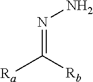

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, “hydroxy” or “hydroxyl” refers to an —OH group.

As used herein, the term “interstitial region” refers to an area, e.g., of a substrate, that separates depressions or protrusions. For example, an interstitial region can separate one depression or protrusion of an array from another depression or protrusion of the array. The two depressions or protrusions that are separated from each other can be discrete, i.e., lacking physical contact with each other. In many examples, the interstitial region is continuous, whereas the depressions or protrusions are discrete, for example, as is the case for a plurality of depressions defined in an otherwise continuous surface or a plurality of protrusions defined on an otherwise continuous surface. Interstitial regions may have a surface material that differs from the surface material of the depressions or protrusions. For example, depressions can have a polymeric hydrogel layer and primers therein, and the interstitial regions can be free of the polymeric hydrogel layer and primers.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

“Nitrone,” as used herein, means a

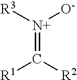

group in which R¹, R², and R³ may be any of the Rₐ and R_{b} groups defined herein, except that R³ is not hydrogen (H).

As used herein, a “nucleotide” includes a nitrogen-containing heterocyclic base, a sugar, and one or more phosphate groups. Nucleotides are monomeric units of a nucleic acid sequence. In RNA, the sugar is a ribose, and in DNA, the sugar is a deoxyribose, i.e. a sugar lacking a hydroxyl group that is present at the 2′ position in ribose. The nitrogen-containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

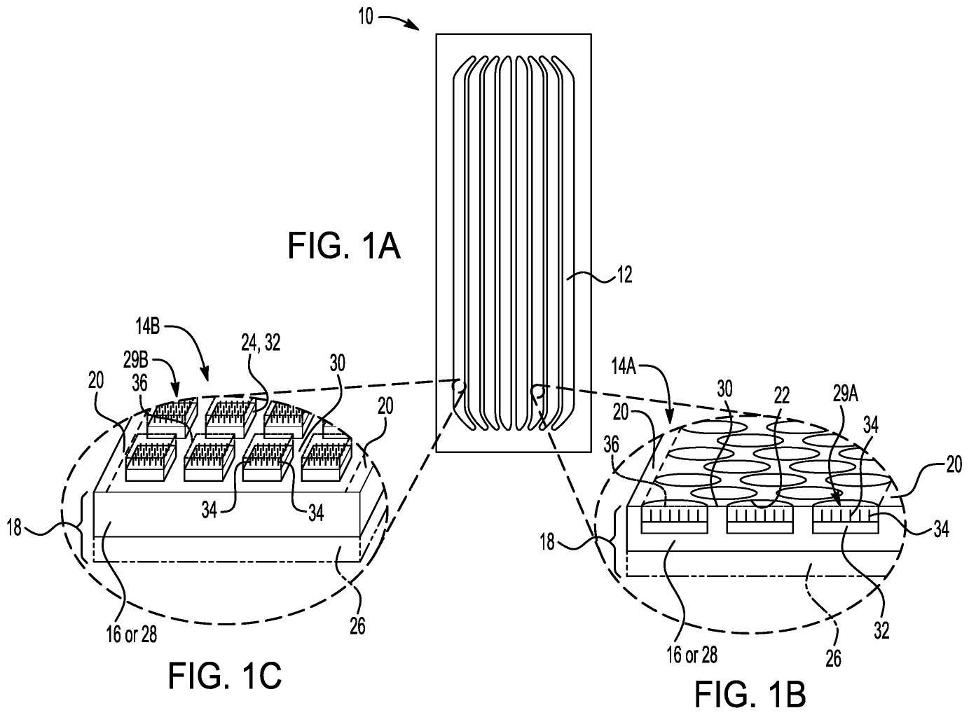

In some examples, the term “over” may mean that one component or material is positioned directly on another component or material. When one is directly on another, the two are in contact with each other. For example, in FIG. 1C, when the multi-layered substrate 18 is included, the plurality of functionalized pads 24 is positioned directly over the layer 28.

In other examples, the term “over” may mean that one component or material is positioned indirectly on another component or material. By indirectly on, it is meant that a gap or an additional component or material may be positioned between the two components or materials. For example, in FIG. 1C, when the multi-layered substrate 18 is included, the plurality of functionalized pads 24 is positioned indirectly over the base support 26. The layer 28 is positioned therebetween.

A “patterned structure” refers to a substrate that includes surface chemistry in a pattern, e.g., in depressions or as protrusions, across the substrate. The surface chemistry may include a polymeric hydrogel layer and primers (e.g., used for library template capture and amplification). In some examples, the substrate has been exposed to patterning techniques (e.g., etching, lithography, etc.) in order to generate the pattern for the surface chemistry. However, the term “patterned structure” is not intended to imply that such patterning techniques have to be used to generate the pattern. The patterned structure may be generated via any of the methods disclosed herein.

As used interchangeably herein, the terms “photo-reactive layer,” “photo-reactive protective layer,” and “photo-reactive coating” refer to a shielding layer (e.g., over a reactive entity) that is capable of undergoing a chemical/physical reaction upon exposure to a predetermined range of light wavelengths, whereby the layer becomes removed or becomes removable using a solvent during or after the reaction. The terms may further refer to materials that are capable of preventing the reactive entity from chemically reacting prior to the removal of the photo-reactive layer.

As used interchangeably herein, the terms “photo-thermal filler” and “photo-thermal additive” refer to nano-sized or micro-sized structures that absorb light at predetermined wavelengths and convert the absorbed energy to heat (thermal energy). The photo-thermal fillers (or photo-thermal additives) described herein may be included as part of a composite including a thermo-responsive polymer and the photo-thermal additive, where the composite is included in an independently removable photo-reactive coating.

As used herein, the term “polyhedral oligomeric silsesquioxane” refers to a chemical composition that is a hybrid intermediate (e.g., RSiO_{1.5}) between that of silica (SiO₂) and silicone (R₂SiO). An example of polyhedral oligomeric silsesquioxane may be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for polyhedral oligomeric silsesquioxane include epoxy, azide/azido, a thiol, a poly(ethylene glycol), a norbornene, a tetrazine, acrylates, and/or methacrylates, or further, for example, alkyl, aryl, alkoxy, and/or haloalkyl groups.

As used herein, a “polymeric hydrogel layer” refers to a gel material that is applied over at least a portion of a substrate. The gel material includes functional group(s) that can attach to primer(s). The polymeric hydrogel layer may be positioned within a portion of a depression defined in the substrate (see the depressions 22 in FIG. 1B), or may define a protrusion on a substrate (see the protrusions 24 in FIG. 1C).

As used herein, the “primer” is defined as a single stranded nucleic acid sequence (e.g., single strand DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of a polymer. The primer length can be any number of bases long and can include a variety of non-natural nucleotides. In an example, the sequencing primer is a short strand, ranging from 10 to 60 bases, or from 20 to 40 bases.

As used herein, the term “protrusion” refers to a discrete convex feature defined on a substrate and surrounded by interstitial region(s) of the substrate. Protrusions can have any of a variety of shapes at their opening in a surface including, as examples, round, elliptical, square, polygonal, star shaped (with any number of vertices), etc. The cross-section of a protrusion taken orthogonally with the surface can be curved, square, polygonal, hyperbolic, conical, angular, etc.

As used herein, the term “reactive entity” refers to the flow cell surface chemistry that enables a desired interaction or reaction. As examples, the reactive entity may be a primer that serves as a starting point for template amplification and cluster generation or an enzyme tag, such as a transposome complex used in tagmentation, or reactive functional groups, such as dibenzocyclooctyne (DBCO), strained alkynes or azides, and biotin.

A “reactive region,” as used herein, refers to the discrete area on or in the substrate that includes the polymeric hydrogel and the reactive entity.

A “removable coating”, a “protective coating”, or an “independently removable coating” is a layer that is positioned over the reactive region and that can be removed from the reactive region without deleteriously affecting the polymeric hydrogel layer of the reactive entity of the reactive region. The coatings described herein may be susceptible to different solvents/removers such that the remover for one type of coating does not remove a different type of coating. These terms may refer to photo-reactive coatings (as defined herein) or coatings having various properties in terms of solubility.

A “remover” is a material or mechanism that is capable of removing a removable coating.

A “spacer layer,” as used herein refers to a material that bonds two components together. In some examples, the spacer layer can be a radiation absorbing material that aids in bonding, or can be put into contact with a radiation absorbing material that aids in bonding.

The term “substrate” refers to the single layer base support or a multi-layer structure upon which the reactive regions are introduced.

The term “thermo-responsive polymer,” as used herein, refers to polymeric materials that are capable of undergoing a phase transition (e.g., melting, transitioning from a hydrophobic state to hydrophilic state, transitioning from a hydrophilic state to hydrophobic state, etc.) when exposed to a certain change in temperature. The thermo-responsive polymer(s) described herein may be included as part of a composite including an example of the thermo-responsive polymer(s) and a photo-thermal additive.

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refer to five-membered heterocyclic group including four nitrogen atoms. Tetrazole can be optionally substituted.

### Flow Cells

Examples of the flow cells disclosed herein generally include a substrate; a plurality of reactive regions spatially separated from one another across the substrate, each of the plurality of reactive regions including: a polymeric hydrogel layer and a reactive entity attached to the polymeric hydrogel layer; and a plurality of independently removable coatings respectively positioned over each of the plurality of reactive regions.

A top view of a flow cell 10 is shown in FIG. 1A, and different examples of the architecture within a flow channel 12 of the flow cell 10 are shown in FIG. 1B and FIG. 1C. Each of the architectures may include one patterned structure 14A or 14B bonded to a lid (not shown) or two patterned structures (the second of which is not shown) bonded together. In still other architectures, the patterned structure 14A, 14B is an open wafer, and thus is not bonded to either a lid or another patterned structure.

In some examples, the flow channel 12 is defined between the one patterned structure 14A or 14B and the lid or the second patterned structure, which are bonded together via a spacer layer (not shown). Thus, in these examples, each flow channel 12 is defined by the patterned structure 14A or 14B, the spacer layer, and either the lid or the second patterned structure. In other examples, the patterned structure 14A or 14B may include sidewalls at the perimeter (e.g., at the bonding region 20) that extend above the interstitial regions 30 to create an open flow channel 12.

The example flow cell 10 shown in FIG. 1A includes eight flow channels 12. While eight flow channels 12 are shown, it is to be understood that any number of flow channels 12 may be included in the flow cell 10 (e.g., a single flow channel 12, four flow channels 12, etc.). When multiple flow channels 12 are included, each flow channel 12 may be isolated from another flow channel 12 so that fluid introduced into a flow channel 12 does not flow into adjacent flow channel(s) 12.

Each flow channel 12 is in fluid communication with an inlet and an outlet (not shown). The inlet and outlet of each flow channel 12 may be positioned at opposed ends of the flow cell 10. The inlets and outlets of the respective flow channels 12 may alternatively be positioned anywhere along the length and width of the flow channel 12 that enables desirable fluid flow.

The inlet allows fluids to be introduced into the flow channel 12, and the outlet allows fluid to be extracted from the flow channel 12. Each of the inlets and outlets is fluidly connected to a fluidic control system (including, e.g., reservoirs, pumps, valves, waste containers, and the like) which controls fluid introduction and expulsion. Some examples of the fluids introduced into the flow channel 12 may introduce reaction components (e.g., DNA sample, polymerases, sequencing primers, nucleotides, etc.), washing solutions, deblocking agents, etc.

The flow channel 12 may have any desirable shape. In an example, the flow channel 12 has a substantially rectangular configuration with curved ends (as shown in FIG. 1A). The length of the flow channel 12 depends, in part, upon the size of the substrate (e.g., 16 or 18, see FIG. 1B and FIG. 1C) used to form the patterned structure 14A or 14B. The width of the flow channel 12 depends, in part, upon the size of the substrate 16 or 18 used to form the patterned structure 14A, 14B, the desired number of flow channels 12, the desired space between adjacent channels 12, and the desired space at a perimeter of the patterned structure 14A or 14B. The spaces between channels 12 and at the perimeter may be sufficient for attachment to a lid (not shown) or another patterned structure (also not shown).

The depth of the flow channel 12 can be as small as a monolayer thick when microcontact, aerosol, or inkjet printing is used to deposit a separate material (e.g., the spacer layer (not shown)) that defines at least a portion of the sidewalls of the flow channel 12. For other examples, the depth of the flow channel 12 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 12 may be greater than, less than or between the values specified above.

The spacer layer used to attach the patterned structure 14A, 14B and the lid or the second patterned structure may be any material that will seal portions of the patterned structure 14A, 14B and the lid or the second patterned structure. As examples, the spacer layer may be an adhesive, a radiation-absorbing material that aids in bonding, or the like. In some examples, the spacer layer is the radiation-absorbing material, e.g., KAPTON® black.

The patterned structure 14A, 14B and the lid or the second patterned structure may be bonded using any suitable technique, such as laser bonding, diffusion bonding, anodic bonding, eutectic bonding, plasma activation bonding, glass frit bonding, or others methods known in the art.

When used, the lid may be any material that is transparent to the excitation light that is directed toward the flow cell 10. In optical detection systems, the lid may also be transparent to the emissions generated from reaction(s) taking place in the flow cell 10. As examples, the lid may include glass (e.g., borosilicate, fused silica, etc.) or a transparent polymer. A commercially available example of a suitable borosilicate glass is D 263®, available from Schott North America Inc. Commercially available examples of suitable polymer materials, namely cyclo olefin polymers, are the ZEONOR® products available from Zeon Chemicals L.P. In some instances, the lid is shaped to form the top of the flow cell 10, and in other instances, the lid is shaped to form both the top of the flow cell as well as sidewalls the flow channel 12.

The patterned structure 14A, 14B includes a bonding region 20 where it can be sealed to the lid or to the second patterned structure. The bonding region 20 may be located at the perimeter of each flow channel 12 (as shown in FIG. 1B and FIG. 1C) and at the perimeter of the flow cell 10. This region 20 may function as the perimeter of the open wafer version of the patterned structure 14A, 14B.

The examples shown in FIGS. 1B and 1C may also be used as open-wafer flow cells without a lid or second patterned structure. In these examples, the patterned structures 14A, 14B are open to the external environment.

The patterned structure 14A, 14B includes a substrate 16 or 18, as shown in FIG. 1B and FIG. 1C. The substrate 16 is a single layer base support, and the substrate 18 is a multi-layered structure. The substrate 16 is a single material that has depressions 22 defined therein or protrusions 24 defined thereon. The substrate 18 includes a base support 26 and another layer 28 positioned on the base support 26, where the other layer 28 has the depressions 22 defined therein or the protrusions 24 defined thereon.

Examples of suitable materials for the substrate 16 include siloxanes (e.g., epoxy siloxane), glass, modified or functionalized glass, polymeric materials (including acrylics, polystyrene and copolymers of styrene and other materials, polypropylene, polyethylene, polybutylene, polyurethanes, polytetrafluoroethylene (such as TEFLON® from Chemours), polyethylene terephthalate (PET), polycarbonate, cyclic olefins/cyclo-olefin polymers (COP) (such as ZEONOR® from Zeon), polyimides, nylon (polyamides), etc.) ceramics/ceramic oxides, aluminum silicate, silicon and modified silicon (e.g., boron doped p+ silicon), silicon nitride (Si₃N₄), carbon, metals, resins, or the like. Examples of suitable inorganic resins include inorganic oxides, such as tantalum pentoxide (e.g., Ta₂O₅) or other tantalum oxide(s) (TaOₓ), aluminum oxide (e.g., Al₂O₃), silica (i.e., silicon dioxide (SiO₂)), fused silica, or silica-based materials, hafnium oxide (e.g., HfO₂), indium tin oxide, titanium dioxide, etc. Examples of suitable polymeric resins include polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As mentioned, examples of the multi-layered structure (i.e., substrate 18) include the base support 26 and at least one other layer 28 thereon. Any example of the single layer base support (i.e., substrate 16) may be used as the base support 26. In these examples, the other layer 28 may be any material that can be etched or imprinted to form the depressions 22. Examples of the layer 28 include inorganic oxides, such as tantalum oxide (e.g., Ta₂O₅), aluminum oxide (e.g., Al₂O₃), silicon oxide (e.g., SiO₂), or hafnium oxide (e.g., HfO₂), or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

In any of the examples set forth herein, the substrate 16 or the base support 26 may be a circular sheet, a panel, a wafer, a die, etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). As one example, a die may have a width ranging from about 0.1 mm to about 10 mm. While example dimensions have been provided, it is to be understood that the substrate 16 or the base support 26 may have any suitable dimensions.

As shown in FIG. 9, one example of the flow cell 10′ includes a complementary metal oxide semiconductor (CMOS) chip 94 that is attached to the substrate 16. For ease of illustration, the substrate 16 is shown in FIG. 9. It is to be understood, however, that the multi-layered substrate 18 could be used instead, and the CMOS chip 94 would be attached to the substrate 18 through the base support 26.

In addition to the complementary metal oxide semiconductor chip 94, this example flow cell 10′ includes the substrate 16 over the complementary metal oxide semiconductor chip 94, the substrate 16 including a plurality of depressions 22 separated by interstitial regions 30; a polymeric hydrogel 32 within the depressions 22; and a reactive entity 34 attached to the polymeric hydrogel 32.

In the illustrated example, the substrate 16 may be affixed directly to, and thus be in physical contact with, the complementary metal oxide semiconductor chip 94 through one or more securing mechanisms (e.g., adhesive, bond, fasteners, and the like). It is to be understood that the substrate 16 may be removably coupled to the complementary metal oxide semiconductor (CMOS) chip 94.

The CMOS chip 94 includes a plurality of stacked layers 96 including, for example, silicon layer(s), dielectric layer(s), metal-dielectric layer(s), metal layer(s), etc.). The stacked layers 96 make up the device circuitry, which includes detection circuitry.

The CMOS chip 94 includes optical components, such as optical sensor(s) 98 and optical waveguide(s) 100. The optical components are arranged such that each optical sensor 98 at least substantially aligns with, and thus is operatively associated with, a single optical waveguide 100 and a single reactive region 29A or 29B of the flow cell 10′. However, in other examples, a single optical sensor 98 may receive photons through more than one optical waveguide 100 and/or from more than one reactive region 29A, 29B. In these other examples, the single optical sensor 98 is operatively associated with more than one optical waveguide 100 and/or more than one reactive region 29A, 29B.

As used herein, a single optical sensor 98 may be a light sensor that includes one pixel or more than one pixel. As an example, each optical sensor 98 may have a detection area that is less than about 50 μm². As another example, the detection area may be less than about 10 μm². As still another example, the detection area may be less than about 2 μm². In the latter example, the optical sensor 98 may constitute a single pixel. An average read noise of each pixel of the optical sensor 98 may be, for example, less than about 150 electrons. In other examples, the read noise may be less than about 5 electrons. The resolution of the optical sensor(s) 98 may be greater than about 0.5 megapixels (Mpixels). In other examples, the resolution may be greater than about 5 Mpixels, or greater than about 10 Mpixels.

Also as used herein, a single optical waveguide 100 may be a light guide including a cured filter material that i) filters the excitation light 104 (propagating from an exterior of the flow cell 10′ into the flow channel 12), and ii) permits the light emissions (not shown, resulting from reactions at the reactive region(s) 29A, 29B) to propagate therethrough toward corresponding optical sensor(s) 98. In an example, the optical waveguide 100 may be, for example, an organic absorption filter. As a specific example, the organic absorption filter may filter excitation light 104 of about 532 nm wavelength and permit light emissions of about 570 nm or more wavelengths. The optical waveguide 100 may be formed by first forming a guide cavity in a dielectric layer 106, and then filling the guide cavity with a suitable filter material.

The optical waveguide 100 may be configured relative to the dielectric material 106 in order to form a light-guiding structure. For example, the optical waveguide 100 may have a refractive index of about 2.0 so that the light emissions are substantially reflected at an interface between the optical waveguide 100 and the surrounding dielectric material 106. In certain examples, the optical waveguide 100 is selected such that the optical density (OD) or absorbance of the excitation light 104 is at least about 4 OD. More specifically, the filter material may be selected and the optical waveguide 100 may be dimensioned to achieve at least 4 OD. In other examples, the optical waveguide 100 may be configured to achieve at least about 5 OD or at least about 6 OD.

In this example, the substrate 16 functions a passivation layer. At least a portion of the passivating substrate 16 is in contact with a first embedded metal layer 112 of the CMOS chip 94 and also with an input region 110 of the optical waveguide 100. The contact between the passivating substrate 16 and the first embedded metal layer 112 may be direct contact or may be indirect contact through a shield layer 114.

The substrate 16 (passivation layer) may provide one level of corrosion protection for the embedded metal layer 112 of the CMOS chip 94 that is closest in proximity to the substrate 16. In this example, the substrate 16 may include a passivation material that is transparent to the light emissions resulting from reactions at the reactive region 29A, 29B (e.g., visible light), and that is at least initially resistant to the fluidic environment and moisture that may be introduced into or present in the flow channel 12. An at least initially resistant material acts as an etch barrier to high pH reagents (e.g., pH ranging from 8 to 14) and as a moisture barrier. Examples of suitable materials for the substrate 16 (or base support 26) of the flow cell 10′ include silicon nitride (Si₃N₄), silicon oxide (SiO₂), tantalum pentoxide (TaO₅), hafnium oxide (HfO₂), boron doped p+ silicon, or the like. The thickness of the substrate 16 may vary depending, in part upon the sensor dimensions. In an example, the thickness of the substrate 16 ranges from about 100 nm to about 500 nm.

In an example, the lid 116 of the flow cell 10′ may be a substantially rectangular block having an at least substantially planar exterior surface 118 and an at least substantially planar interior surface 120 that defines a portion of the flow channel 12. The block may be mounted onto the material 64. Alternatively, the block may be etched so that the lid 116 defines both the top and sidewalls of the channel 12. In these instances, a thin layer of the material 64 may bind the lid 116 to the substrate 16. In an example, a recess may be etched into the transparent block. When the etched block is mounted to the substrate 16, the recess may become the flow channel 12.

As noted, the lid 116 may be physically connected to the substrate 16 through material 64. The material 64 is/are coupled to a portion the surface of the substrate 16 and extends between those portions of the lid 116 that are to contact the substrate 16. In some examples, the material 64 includes a curable adhesive layer that bonds the lid 116 to the substrate 16 (at a portion of its surface).

The lid 116 may include inlet and outlet ports 122, 124 that are configured to fluidically engage other ports (not shown) for directing fluid(s) into the flow channel 12 (e.g., from a reagent cartridge or other fluid storage system component) and out of the flow channel 12 (e.g., to a waste removal system).

The flow channel 12 may be sized and shaped to direct a fluid along the reactive region(s) 29A, 29B. The height of the flow channel 12 and other dimensions of the flow channel 12 may be configured to maintain a substantially even flow of the fluid along the reactive region(s) 29A, 29B. The dimensions of the flow channel 12 may also be configured to control bubble formation. In an example, the height of the flow channel 12 may range from about 50 μm to about 400 μm. In another example, the height of the flow channel 12 may range from about 80 μm to about 200 μm. It is to be understood that the height of the flow channel 12 may vary, and may be the greatest when the reactive region 29A, 29B is located in a reaction chamber (e.g., depression 22) that is defined in the surface of the substrate 16. In these examples, the depression 22 increases the height of the flow channel 12 at this particular area.

Each reactive region 29A, 29B is a localized region in the substrate 16 where a designated reaction may occur. In the flow cell 10′, each reactive region 29A, 29B is a depression 22 having the polymeric hydrogel 32 therein, where polymeric hydrogel 32 is functionalized with the reactive entity 34. While not depicted in FIG. 9, each of the reactive regions 29A, 29B may include an example of the independently removable coating 36 disclosed herein overlying the reactive entities 34.

The polymeric hydrogel 32 may be selectively deposited (e.g., using sputter coating, thermal evaporation, or any other suitable technique) into the depressions 22, or applied and polished as described herein in reference to FIG. 1B.

In an example, the reactive region 29A or 29B is at least substantially aligned with the input region 110 of a single optical waveguide 100. As such, light emissions at the reactive region 29A, 29B may be directed into the input region 110, through the waveguide 100, and to an associated optical sensor 98. In other examples, one reactive region 29A, 29B may be aligned with several input regions 110 of several optical waveguides 100. In still other examples, several reactive regions 29A, 29B may be aligned with one input region 110 of one optical waveguide 100.

The embedded metal layer 112 may be any suitable CMOS metal, such as aluminum (Al), aluminum chloride (AlCl), tungsten (W), nickel (Ni), or copper (Cu). The embedded metal layer 112 is a functioning part of the CMOS AVdd line, and through the stacked layers 96, is also electrically connected to the optical sensor 98. Thus, the embedded metal layer 112 participates in the detection/sensing operation.

It is to be understood that the other optical sensors 98 and associated components may be configured in an identical or similar manner. It is also to be understood, however, that the CMOS chip 94 may not be manufactured identically or uniformly throughout. Instead, one or more optical sensor 98 and/or associated components may be manufactured differently or have different relationships with respect to one another

The stacked layer 96 may include interconnected conductive elements (e.g., conductors, traces, vias, interconnects, etc.) that can conduct electrical current. The circuitry may be configured for selectively transmitting data signals that are based on detected photons. The circuitry may also be configured for signal amplification, digitization, storage, and/or processing. The circuitry may collect and analyze the detected light emissions and generate data signals for communicating detection data to a bioassay system. The circuitry may also perform additional analog and/or digital signal processing in the CMOS chip 94.

The CMOS chip 94 may be manufactured using integrated circuit manufacturing processes. The CMOS chip 94 may include multiple layers, such as a sensor base/layer (e.g., a silicon layer or wafer). The sensor base may include the optical sensor 98. When the CMOS chip 94 is fully formed, the optical sensor 98 may be electrically coupled to the rest of the circuitry in the stack layer 96 through gate(s), transistor(s), etc.

As used in reference to FIG. 9, the term “layer” is not limited to a single continuous body of material unless otherwise noted. For example, the sensor base/layer may include multiple sub-layers that are different materials and/or may include coatings, adhesives, and the like. Furthermore, one or more of the layers (or sub-layers) may be modified (e.g., etched, deposited with material, etc.) to provide the features described herein.

The stacked layer 96 also includes a plurality of metal-dielectric layers. Each of these layers includes metallic elements (e.g., M1-M5, which may be, for example, W (tungsten), Cu (copper), Al (aluminum), or any other suitable CMOS conductive material) and dielectric material 106 (e.g., SiO₂). Various metallic elements M1-M5 and dielectric materials 106 may be used, such as those suitable for integrated circuit manufacturing.

In the example shown in FIG. 9, each of the plurality of metal-dielectric layers L1-L6 includes both metallic elements M1, M2, M3, M4, M5 and dielectric material 106. In each of the layers L1-L6, the metallic elements M1, M2, M3, M4, M5 are interconnected and are embedded within dielectric material 106. In some of the metal-dielectric layers L1-L6, additional metallic elements may also be included. Some of these additional metallic elements may be used to address individual pixels through a row and column selector. The voltages at these elements may vary and switch between about −1.4 V and about 4.4 V depending upon which pixel the device is reading out.

The configuration of the metallic elements M1, M2, M3, M4, M5 and dielectric layer 106 in FIG. 9 is illustrative of the circuitry, and it is to be understood that other examples may include fewer or additional layers and/or may have different configurations of the metallic elements M1-M5.

In the example shown in FIG. 9, the shield layer 114 is in contact with at least a portion of the substrate 16. The shield layer 114 has an aperture at least partially adjacent to the input region 110 of the optical waveguide 100. This aperture enables the reactive region 29A, 29B (and at least some of the light emissions therefrom) to be optically connected to the waveguide 100. It is to be understood that the shield layer 114 may have an aperture at least partially adjacent to the input region 110 of each optical waveguide 100. The shield layer 114 may extend continuously between adjacent apertures.

The shield layer 114 may include any material that can block, reflect, and/or significantly attenuate the light signals that are propagating through the flow channel 12. The light signals may be the excitation light 104 and/or the light emissions from the reactive region(s) 29A, 29B. As an example, the shield layer 114 may be tungsten (W).

It is to be understood that the flow cell 10′ may also be used for optical detection.

Returning now to FIG. 1B and FIG. 1C, different examples of the architecture within the flow channel 12 of the flow cell 10 are respectively depicted. In FIG. 1B, the depressions 22 are defined in the substrate 16 or in the layer 28 of the substrate 18. In FIG. 1C, the protrusions 24 are defined on the substrate 16 or on the layer 28 of the substrate 18.

Many different layouts of the depressions 22 or protrusions 24 may be envisaged, including regular, repeating, and non-regular patterns. In an example, the depressions 22 or protrusions 24 are disposed in a hexagonal grid for close packing and improved density. Other layouts may include, for example, rectangular layouts, triangular layouts, and so forth. In some examples, the layout or pattern can be an x-y format in rows and columns. In some other examples, the layout or pattern can be a repeating arrangement of the depressions 22 or protrusions 24 and interstitial regions 30. In still other examples, the layout or pattern can be a random arrangement of the depressions 22 or protrusions 24 and the interstitial regions 30.

The layout or pattern may be characterized with respect to the density (number) of the depressions 22 or protrusions 24 in a defined area. For example, the depressions 22 or protrusions 24 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high density array may be characterized as having the depressions 22 or protrusions 24 separated by less than about 100 nm, a medium density array may be characterized as having the depressions 22 or protrusions 24 separated by about 400 nm to about 1 μm, and a low density array may be characterized as having the depressions 22 or protrusions 24 separated by greater than about 1 μm.

The layout or pattern of the depressions 22 or protrusions 24 may also or alternatively be characterized in terms of the average pitch, or the spacing from the center of one depression 22 or protrusion 24 to the center of an adjacent depression 22 or protrusion 24 (center-to-center spacing) or from the right edge of one depression 22 or protrusion 24 to the left edge of an adjacent depression 22 or protrusion 24. The pattern can be regular, such that the coefficient of variation around the average pitch is small, or the pattern can be non-regular in which case the coefficient of variation can be relatively large. In either case, the average pitch can be, for example, about 50 nm, about 0.15 μm, about 0.5 μm, about 1 μm, about 5 μm, about 10 μm, about 100 μm, or more or less. The average pitch for a particular pattern of depressions 22 or protrusions 24 can be between one of the lower values and one of the upper values selected from the ranges herein. In an example, the depressions 22 or protrusions 24 have a pitch (center-to-center spacing) of about 1.5 μm. While example average pitch values have been provided, it is to be understood that other average pitch values may be used.

The size of each depression 22 may be characterized by its volume, opening area, depth, and/or diameter or length and width. For example, the volume can range from about 1×10⁻³ μm³ to about 100 μm³, e.g., about 1×10⁻² μm³, about 0.1 μm³, about 1 μm³, about 10 μm³, or more, or less. For another example, the opening area can range from about 1×10⁻³ μm² to about 100 μm², e.g., about 1×10⁻² μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less.

The size of each protrusion 24 may be characterized by its top surface area, height, and/or diameter or length and width. The top surface area can range from about 1×10⁻³ μm² to about 100 μm², e.g., about 1×10⁻² μm², about 0.1 μm², about 1 μm², at least about 10 μm², or more, or less. The height can range from about 0.1 μm to about 100 μm, e.g., about 0.5 μm, about 1 μm, about 10 μm, or more, or less. The diameter or each of the length and the width can range from about 0.1 μm to about 100 μm, e.g., about 0.5 μm, about 1 μm, about 10 μm, or more, or less.

Each of the architectures also includes the reactive regions 29A or 29B. In the example shown in FIG. 1B, the reactive region 29A includes the polymeric hydrogel 32 applied within the depressions 22 and the reactive entity 34 attached to the polymeric hydrogel 32. In the example shown in FIG. 1C, the reactive region 29B includes the polymeric hydrogel 32 applied on the substrate 16 or layer 28 in the form of the protrusions 24 and the reactive entity 34 attached to the polymeric hydrogel 32.

The polymeric hydrogel 32 may be any gel material that can swell when liquid is taken up and that can contract when liquid is removed, e.g., by drying. In an example, the gel material is an acrylamide copolymer. Some examples of the acrylamide copolymer are represented by the following structure (I):

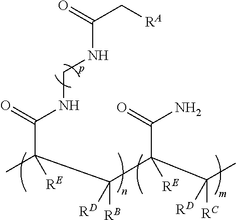

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

One specific example of the acrylamide copolymer represented by structure (I) is poly(N-(5-azidoacetamidylpentyl)acrylamide-co-acrylamide), PAZAM.

One of ordinary skill in the art will recognize that the arrangement of the recurring “n” and “m” features in structure (I) are representative, and the monomeric subunits may be present in any order in the polymer structure (e.g., random, block, patterned, or a combination thereof). The molecular weight of the acrylamide copolymer may range from about 5 kDa to about 1500 kDa or from about 10 kDa to about 1000 kDa, or may be, in a specific example, about 312 kDa.

In some examples, the acrylamide copolymer is a linear polymer. In some other examples, the acrylamide copolymer is a lightly cross-linked polymer.

In other examples, the gel material may be a variation of structure (I). In one example, the acrylamide unit may be replaced with N,N-dimethylacrylamide

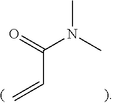

In this example, the acrylamide unit in structure (I) may be replaced with,

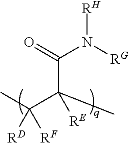

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

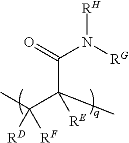

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the gel material, the recurring “n” feature in structure (I) may be replaced with a monomer including a heterocyclic azido group having structure (II):

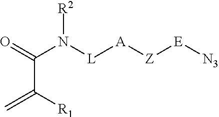

wherein R¹ is H or a C1-C6 alkyl; R² is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the gel material may include a recurring unit of each of structure (III) and (IV):

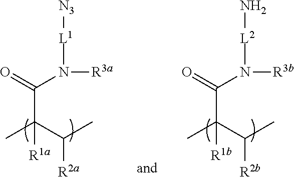

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

In still another example, the acrylamide copolymer is formed using nitroxide mediated polymerization, and thus at least some of the copolymer chains have an alkoxyamine end group. In the copolymer chain, the term “alkoxyamine end group” refers to the dormant species —ONR₁R₂, where each of R₁ and R₂ may be the same or different, and may independently be a linear or branched alkyl, or a ring structure, and where the oxygen atom is attached to the rest of the copolymer chain. In some examples, the alkoxyamine may also be introduced into some of the recurring acrylamide monomers, e.g., at position R^{A} in structure (I). As such, in one example, structure (I) includes an alkoxyamine end group; and in another example, structure (I) includes an alkoxyamine end group and alkoxyamine groups in at least some of the side chains.

It is to be understood that other molecules may be used to form the polymeric hydrogel 32, as long as they are capable of being functionalized with the desired chemistry, e.g., primer set(s) or another desired reactive entity 34. Some examples of suitable materials for the polymeric hydrogel 32 include functionalized silanes, such as norbornene silane, azido silane, alkyne functionalized silane, amine functionalized silane, maleimide silane, or any other silane having functional groups that can respectively attach the desired chemistry. Still other examples of suitable materials for the polymeric hydrogel 32 include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable materials for the polymeric hydrogel 32 include mixed copolymers of acrylamides and acrylates. A variety of polymer architectures containing acrylic monomers (e.g., acrylamides, acrylates etc.) may be utilized in the examples disclosed herein, such as branched polymers, including dendrimers (e.g., multi-arm or star polymers or star-block polymers). For example, the monomers (e.g., acrylamide, acrylamide containing the catalyst, etc.) may be incorporated, either randomly or in block, into the branches (arms) of a dendrimer.

The gel material for the polymeric hydrogel 32 may be formed using any suitable copolymerization process, such as nitroxide mediated polymerization (NMP), reversible addition-fragmentation chain-transfer (RAFT) polymerization, etc.

The attachment of the polymeric hydrogel 32 to the underlying component (e.g., substrate 16 or layer 28) may be through covalent bonding. In some instances, the underlying substrate 16 or layer 28 may first be activated, e.g., through silanization or plasma ashing. Covalent linking is helpful for maintaining the primer set in the desired regions throughout the lifetime of the flow cell 10 during a variety of uses.

In each example, the polymeric hydrogel 32 has a reactive entity 34 attached thereto. In some examples of the flow cell 10, the reactive entity 34 in each of the plurality of reactive regions 29A, 29B is a primer set. In one of these examples, the primer set is the same in each of the plurality of reactive regions 29A, 29B. In other of these examples, the primer set of at least one of the plurality of reactive regions 29A, 29B is different than the primer set of at least one other of the plurality of reactive regions 29A, 29B.

The primer set that may be used as the reactive entity 34 includes two different primers that are used in sequential paired-end sequencing. In another example, the reactive entity 34 is an enzyme tag, such as a transposome complex that enables tagmentation. As such, in one example, the reactive entity 34 in each of the plurality of reactive regions 29A, 29B is independently selected from the group consisting of a primer set and an enzyme tag.

As mentioned, when the reactive entity 34 is the primer set, the primer set includes two different primers that are used in sequential paired end sequencing. As examples, the primer set may include P5 and P7 primers, P15 and P7 primers, or any combination of the PA primers, the PB primers, the PC primers, and the PD primers set forth herein. As examples, the primer set may include any two PA, PB, PC, and PD primers, or any combination of one PA primer and one PB, PC, or PD primer, or any combination of one PB primer and one PC primer or PD primer, or any combination of one PC primer and one PD primer.

Examples of P5 and P7 primers are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, ISEQ™, GENOME ANALYZER™, and other instrument platforms. The P5 primer (shown as a cleavable primer due to the cleavable nucleobase uracil or “n”) is:

P5 #1: 5′ → 3′
(SEQ. ID. NO. 1)
AATGATACGGCGACCACCGAGAUCTACAC;
P5 #2: 5′ → 3′
(SEQ. ID. NO. 2)
AATGATACGGCGACCACCGAGAnCTACAC
where “n” is inosine in SEQ. ID. NO. 2;
or
P5 #3: 5′ → 3′
(SEQ. ID. NO. 3)
AATGATACGGCGACCACCGAGAnCTACAC

where “n” is alkene-thymidine (i.e., alkene-dT) in SEQ. ID. NO. 3.
The P7 primer (shown as cleavable primers) may be any of the following:

P7 #1: 5′ → 3′
(SEQ. ID. NO. 4)
CAAGCAGAAGACGGCATACGAnAT
where “n” is 8-oxoguanine in SEQ. ID. NO. 4;
P7 #2: 5′ → 3′
(SEQ. ID. NO. 5)
CAAGCAGAAGACGGCATACnAGAT
where “n” is 8-oxoguanine in SEQ. ID. NO. 5;
P7 #3: 5′ → 3′
(SEQ. ID. NO. 6)
CAAGCAGAAGACGGCATACnAnAT
where both instances of “n” are 8-oxoguanine
in SEQ. ID. NO. 6;
P7 #4: 5′ → 3′
(SEQ. ID. NO. 7)
CAAGCAGAAGACGGCATACGAUAT;
or
P7 #5: 5′ → 3′
(SEQ. ID. NO. 8)
CAAGCAGAAGACGGCATACUAGAT.

The P15 primer (shown as a cleavable primer) is:

P15: 5′ → 3′
(SEQ. ID. NO. 9)
AATGATACGGCGACCACCGAGAnCTACAC

where “n” is allyl-T (i.e., a thymine nucleotide analog having an allyl functionality).
The other primers (PA-PD, shown as non-cleavable primers) mentioned above include:

PA 5′ → 3′
(SEQ. ID. NO. 10)
GCTGGCACGTCCGAACGCTTCGTTAATCCGTTGAG
PB 5′ → 3′
(SEQ. ID. NO. 11)
CGTCGTCTGCCATGGCGCTTCGGTGGATATGAACT
PC 5′ → 3′
(SEQ. ID. NO. 12)
ACGGCCGCTAATATCAACGCGTCGAATCCGCAACT,
and
PD 5′ → 3′
(SEQ. ID. NO. 13)
GCCGCGTTACGTTAGCCGGACTATTCGATGCAGC.

While not shown in the example sequences for PA-PD, it is to be understood that any of these primers may include a cleavage site, such as uracil, 8-oxoguanine, allyl-T, etc. at any point in the strand.

Each of the primers disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The 5′ end of each primer may also include a linker. Any linker that includes a terminal alkyne group or another suitable terminal functional group that can attach to the surface functional groups of the polymeric hydrogel 32 may be used. In one example, the primers are terminated with hexynyl functional groups.

As described, in some examples, the same reactive entity 34 (e.g., primer set) is attached to the polymeric hydrogel 32 that is present in each of the depressions 22 or that forms each of the protrusions 24. For example, the primer set is the same in each of the plurality of reactive regions 29A, 29B. In other examples, one reactive entity 34 (e.g., a primer set including P5 and P7 primers) is attached in a subset of the depressions 22 or to a subset of the protrusions 24, while a different reactive entity 34 (e.g., another primer set including PA and PB primers) is attached in a subset of the depressions 22 or to a subset of the protrusions 24. As such, as described, the primer set of at least one of the plurality of reactive regions 29A, 29B is different than the primer set of at least one other of the plurality of reactive regions 29A, 29B.

The architecture shown in FIG. 1B may be generated by forming the depressions 22 in the substrate 16 or the layer 28 of the substrate 18, introducing the polymeric hydrogel 32 into the depressions 22, and attaching the reactive entity 34 (e.g., primers of a primer set) to the polymeric hydrogel 32.

The depressions 22 may be formed using etching or nanoimprint lithography.

A mixture of the polymeric hydrogel 32 may be generated. In one example, the polymeric hydrogel 32 may be present in a mixture (e.g., with water or with ethanol and water). The polymeric hydrogel 32 may be blanketly deposited over the substrate 16 or the layer 28 of the substrate 18, and then removed from the interstitial regions 30 using a polishing technique.

The reactive entity 34 may then be attached to the polymeric hydrogel 32. As an example, primers or transposome complexes may be grafted to the polymeric hydrogel 32. Grafting may be accomplished by flow through deposition (e.g., using a temporarily bound lid), dunk coating, spray coating, puddle dispensing, or by another suitable method. Each of these example techniques may utilize a solution or mixture, which may include the reactive entity 34, water, a buffer, and a catalyst. With any of the grafting methods, the reactive entity 34 attaches to the reactive groups of the polymeric hydrogel 32 and does not react with the interstitial regions 30. As another example, enzyme tags may be attached through oligonucleotide hybridization or through biotin/streptavidin interactions. With oligonucleotide hybridization, the enzyme tag may include an oligonucleotide sequence that can hybridize to polymeric hydrogel bound primers. With biotin/streptavidin interactions, the polymeric hydrogel 32 may be biotinylated, and the enzyme tag may include streptavidin.

When a single type of reactive entity 34 is used, the reactive entity 34 may be pre-grafted to the polymeric hydrogel 32, and the pre-grafted hydrogel may be deposited and polished, or selectively deposited.

When multiple reactive entities 34 are used, some depressions 22 may be masked (e.g., with a photoresist or other suitable mask) while other depressions 22 have one type of reactive entity 34 grafted thereto. Alternatively, the reactive entities 34 may be pre-grafted to different samples of polymeric hydrogel 32 and the pre-grafted hydrogels may be respectively and selectively deposited into the desired depressions 22. High precision coating techniques may be used for the selective deposition. In one example, a precision gantry tool is used.

The architecture shown in FIG. 1C may be generated by forming the protrusions 24 on the substrate 16 or the layer 28 of the substrate 18 using the polymeric hydrogel 32, and attaching the reactive entity 34 to the polymeric hydrogel 32. A mixture of the polymeric hydrogel 32 may be generated as described herein.

In one example, a photoresist may first be deposited on the substrate 16 or the layer 28, and developed such that soluble photoresist portions are removed where it is desirable to form the protrusions 24 and insoluble photoresist portions remain where it is desirable to form the interstitial regions 30. The mixture including the polymeric hydrogel 32 may be blanketly deposited over the insoluble photoresist portions and over the exposed portions of the substrate 16 or the layer 28, and cured. The polymeric hydrogel 32 applied to the exposed portions of the substrate 16 or the layer 28 become the protrusions 24. The insoluble photoresist portions, and the polymeric hydrogel 32 thereon, may be removed using a suitable remover for the photoresist to expose the interstitial regions 30.

Alternatively, the mixture including the polymeric hydrogel 32 may be selectively deposited (using a mask to cover interstitial regions 30, controlled printing techniques, etc.) to specifically deposit the polymeric hydrogel 32 at areas where it is desirable to form the protrusions 24.

The reactive entity 34 may then be grafted to the protrusions 24. As examples, grafting may be accomplished by flow through deposition (e.g., using a temporarily bound lid), dunk coating, spray coating, puddle dispensing, or by another suitable method. Each of these example techniques may utilize a reactive entity 34 solution or mixture, which may include the reactive entity 34, water, a buffer, and/or a catalyst. With any of the grafting methods, the reactive entity 34 attaches to the reactive groups of the polymeric hydrogel 32/protrusions 24 and does not react with the interstitial regions 30.

When a single reactive entity 34 is used, the reactive entity 34 may be pre-grafted to the polymeric hydrogel 32, and the pre-grafted hydrogel may be deposited to form the protrusions 24 according to the examples set forth herein.

When multiple reactive entities 34 are used, some protrusions 24 may be masked (e.g., with a photoresist or other suitable mask) while other protrusions 24 have one type of reactive entity 34 grafted thereto. Alternatively, the reactive entities 34 may be pre-grafted to different samples of polymeric hydrogel 32 and the pre-grafted hydrogels may be respectively and selectively deposited to form the protrusions 24.

Each of the flow cell architectures includes the independently removable coating(s) 36 positioned over the reactive regions 29A, 29B. For clarity, one independently removable coating 36 is shown in each of FIG. 1B and FIG. 1C. The independently removable coating(s) 36 render the reactive regions 29A, 29B inactive (e.g., incapable of participating in a designated chemical reaction) until the independently removable coating(s) 36 overlying the particular reactive region 29A, 29B is/are removed to expose the reactive region 29A, 29B. As such, the coating(s) 36 can be designed so that (a) particular reactive region(s) 29A, 29B is/are exposed for analysis or for a designated reaction at a particular time.

In some examples, the substrate 16, 18 includes a plurality of depressions 22; each of the plurality of reactive regions 29A, 29B is positioned within a respective one of the plurality of depressions 22; and each of the plurality of independently removable coatings 36 covers a respective one of the plurality of reactive regions 29A, 29B. One of these examples is shown in FIG. 1B.

In some other examples, the substrate 16, 18 includes a plurality of protrusions 24; each of the plurality of reactive regions 29A, 29B is positioned at a respective one of the plurality of protrusions 24; and each of the plurality of independently removable coatings 36 covers a respective one of the plurality of protrusions 24. One of these examples is shown in FIG. 1C.

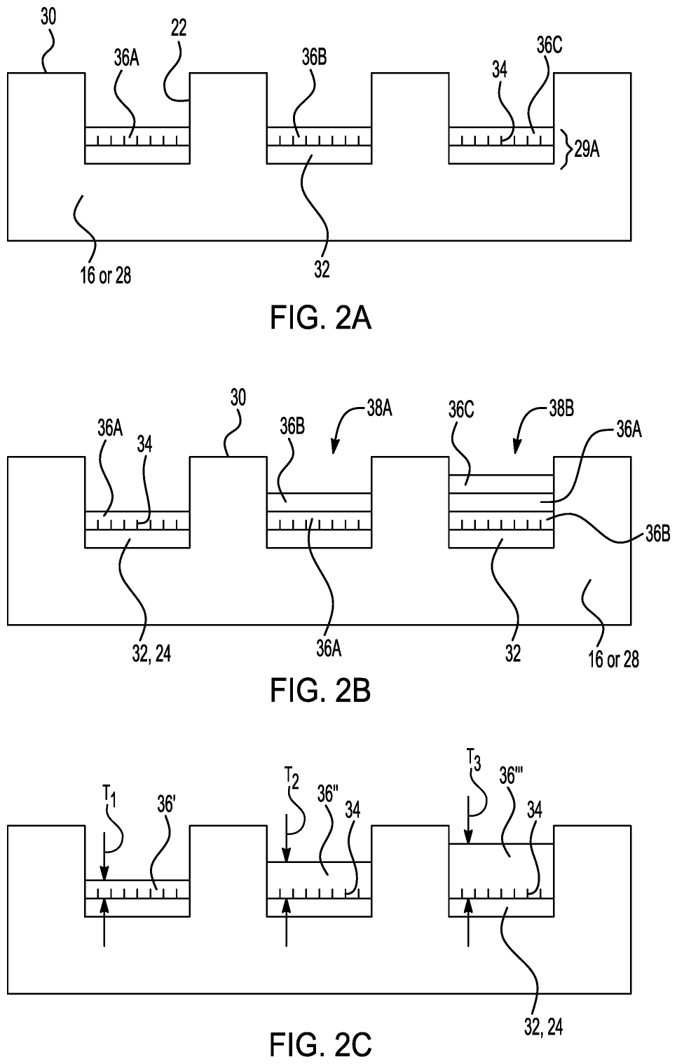

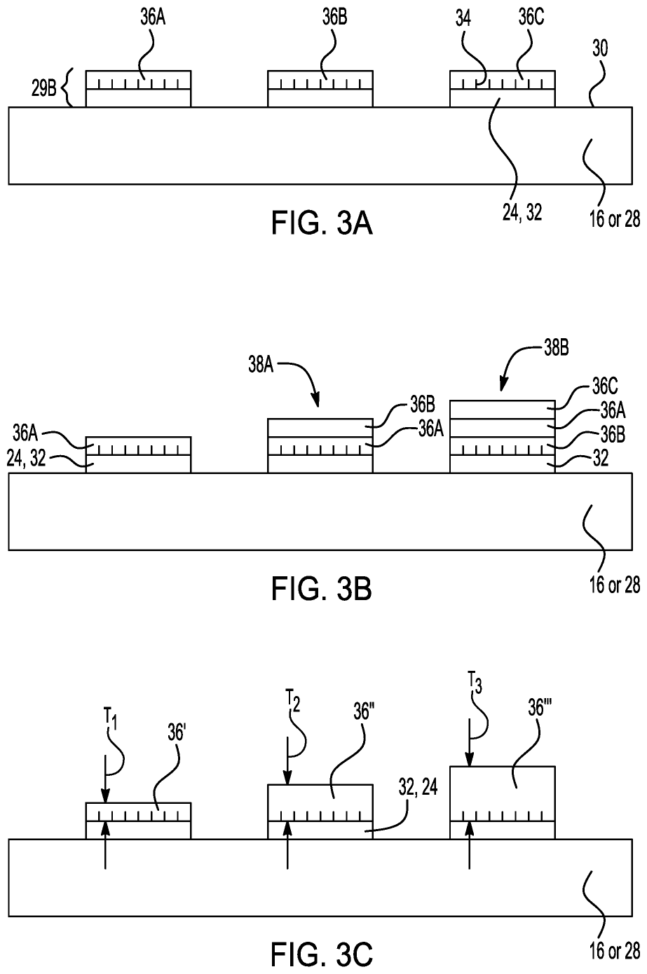

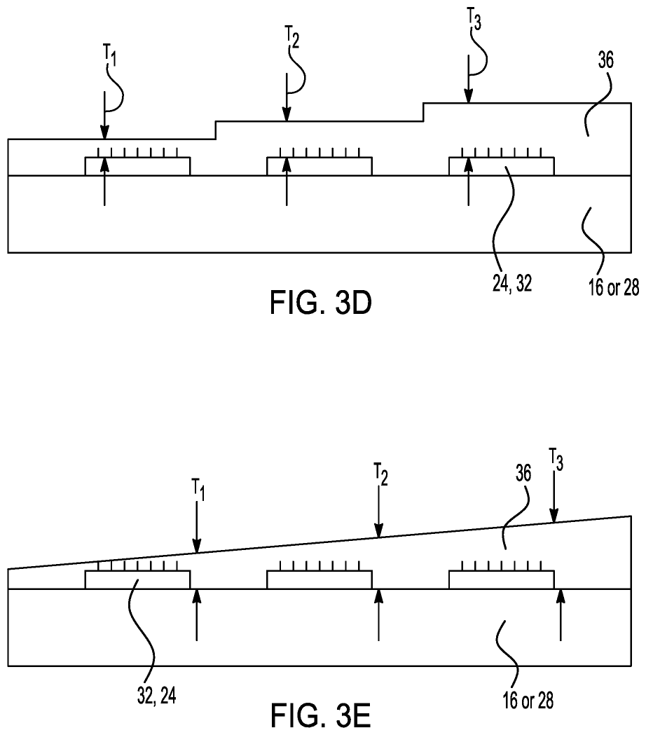

Several different examples of the removable coatings 36 will now described in reference to FIG. 2A through FIG. 2C and FIG. 3A through FIG. 3E.

In some example flow cells 10, at least one of the plurality of independently removable coatings 36 has a different removal characteristic than at least one other of the plurality of independently removable coatings 36. Some examples of these independently removable coatings 36A, 36B, 36C are shown in FIG. 2A and FIG. 3A.

In these examples, the independently removable coatings 36A, 36B, 36C are positioned directly over the reactive regions 29A, 29B and are not positioned on the interstitial regions 30 separating the reactive regions 29A, 29B. While single coatings 36A, 36B, 36C are shown covering single reactive regions 29A, 29B, it is to be understood that each coating 36A, 36B, 36C may cover a subset of reactive regions 29A, 29B, where each subset includes two or more reactive regions 29A, 29B. In these examples, the coating 36A, 36B, 36C may overlie each of the reactive regions 29A, 29B in the subset, as well as the interstitial regions 30 separating the particular reactive regions 29A, 29B. In an example, the coatings 36A, 36B, 36C may each cover from about 1 mm² to about 500 mm² of the substrate surface area.

The respective coatings 36A, 36B, 36C may be applied in the depressions 22 (FIG. 2A) or over the protrusions 24 (FIG. 3A) using selective deposition techniques, such as inkjet printing, aerosol printing, screen printing, or precision dispensing methods (such as slot-die coating). In one example, slot-die coating manifolds can coat rectangular stripes down a web of material in pre-defined thicknesses. By employing a vision system registering the web position, the coating can be turned on and off to create the individual coatings 36A, 36B, 36C. The respective coatings 36A, 36B, 36C may alternatively be applied in the depressions 22 (FIG. 2A) or over the protrusions 24 (FIG. 3A) using blanket deposition techniques (e.g., spray coating) along with removable masks (e.g., photoresists) that cover areas that are not to receive a particular coating 36A, 36B, or 36C being deposited. In still other examples, roll-to-roll deposition methods may be used to transfer coatings from a source to the desired position on the patterned structure 14A, 14B.

In the examples shown in FIG. 2A and FIG. 3A, each of the plurality of independently removable coatings 36A, 36B, 36C has a different removal characteristic with respect to each other of the plurality of independently removable coatings 36A, 36B, 36C. It is to be understood, however, that many combinations and arrangements of the coatings 36A, 36B, 36C are possible. As such, in an array of depressions 22 or protrusions 24, each depression 22 or protrusion 24 in the array can have a different independently removable coating 36A, 36B, 36C thereon, or subsets of depressions 22 or protrusions 24 in the array can have different independently removable coatings 36A, 36B, 36C thereon. Each subset may include two or more depressions 22 or protrusions 24.

One example of the removal characteristic that the coatings 36A, 36B, 36C may exhibit is solubility. In the example shown in FIG. 2A and FIG. 3A, the solubility of one coating 36A is orthogonal to each of the other coatings 36B and 36C. In this context, the term “orthogonal” means that the solubility condition(s) that one coating 36A is susceptible to is at least partially different than the solubility condition(s) that each other coating 36B, 36C is/are susceptible to. The coatings 36A, 36B, 36C may be soluble in different solvents, or may be soluble in different aqueous solutions having different pH values.

Table 1 illustrates one example of completely orthogonal solvents.

TABLE 1

| | Solvent 1 | Solvent 2 | Solvent 3 |
|---|---|---|---|
| Polymer for | Soluble | Insoluble | Insoluble |
| Coating 36A | | | |
| Polymer for | Insoluble | Soluble | Insoluble |
| Coating 36B | | | |
| Polymer for | Insoluble | Insoluble | Soluble |
| Coating 36C | | | |

In this example, three different polymers (that respectively form each of the coatings 36A, 36B, 36C) are susceptible to removal by three different solvents. It is to be understood that any number of polymers may be used for the coatings 36A, 36B, 36C, as long as the desired number of reactive regions 29A, 29B is exposed at a desirable time. For example, half of the reactive regions 29A, 29B along a flow cell channel 12 may be coated with coating 36A and the other half of the reactive regions 29A, 29B may be coated with coating 36B. This enables one half of the reactive regions 29A, 29B to be analyzed (or used in a designated reaction) at one time, and the other half of the reactive regions 29A, 29B to be analyzed (or used in a designated reaction) at another time.

It is to be understood that polymers with partial orthogonality could be used for the coatings 36A, 36B, 36C, as long as the removal of the respective coatings 36A, 36B, 36C is performed in a particular sequence so that reactive regions 29A, 29B are not prematurely exposed. Table 2 illustrates one example of partially orthogonal solvents.

TABLE 2

| | Solvent 1 | Solvent 2 | Solvent 3 |
|---|---|---|---|
| Polymer for | Soluble | Soluble | Soluble or |
| Coating 36A | | | Insoluble |
| Polymer for | Insoluble | Soluble | Insoluble |
| Coating 36B | | | |
| Polymer for | Insoluble | Insoluble | Soluble |
| Coating 36C | | | |

Examples of polymers with partially orthogonal solvent solubility that may be used as the coatings 36A, 36B, 36C, respectively, include acrylic polymers, such as poly(dimethylsiloxane) (PDMS) which is soluble in hexane and toluene; poly(methyl methacrylate) (PMMA), which is insoluble in hexane but soluble in toluene; and poly(vinyl alcohol) (PVA), which is soluble in water but not in hexane or toluene. In this particular example, coating 36A can be removed first with solvent 1 because it will not remove either of the other coatings 36B, 36C. In this particular example, solvent 2 would not be used before solvent 1 unless it would be desirable to expose the reactive regions 29A, 29B covered by each of the coatings 36A, 36B, as solvent 2 would remove both the coatings 36A, 36B.

Several examples of polymers that may be used as the coatings 36A, 36B, 36C, and their solubility in three different solvents are shown in Table 3. Any combination of these polymers may be used for the coatings 36A, 36B, 36C, and two or more of the coatings 36A, 36B, 36C may be used in any channel 12 of the flow cell 10. Moreover, the polymers that are selected for the coatings 36A, 36B, 36C may have partial orthogonality, and the order in which the solvents are used may be selected so that one or more coatings are not prematurely removed while other coatings are removed.

TABLE 3

| | | Aliphatic | |
|---|---|---|---|
| | | alcohol | |
| Polymer type | Water | (ethanol) | Acetone |
| Polyvinyl alcohol (PVA) | Soluble | Insoluble | Insoluble |
| PVA-polyethylene glycol | Soluble | Insoluble | Insoluble |
| (PVA-PEG copolymer) | | | |
| Hydroxypropyl methyl cellulose | Soluble | Soluble | Soluble |
| (HPMC) | | | |
| Hydroxyethyl cellulose (HEC) | Soluble | Soluble | Soluble |
| Hydroxypropyl cellulose (HPC) | Soluble | Soluble | Soluble |
| Polyvinyl acetate | Insoluble | Soluble | Soluble |
| Ethyl cellulose | Insoluble | Soluble | Soluble |
| Ammonio methacrylate | Insoluble | Soluble | Soluble |
| Ammonio methacrylate | Insoluble | Soluble | Soluble |
| copolymer | | | |
| (type A and type B) | | | |
| Cellulose acetate | Insoluble | Insoluble | Soluble |
| Poly(ethyl acrylate-co-methyl | Insoluble | Insoluble | Soluble |
| methacrylate) 2:1 | | | |

Examples of polymers that are soluble in aqueous solutions having different pH levels include chitosan, which is soluble under acidic conditions, and copolymers of non-water soluble monomers with amine functional monomers, which are tunable to dissolve at different pH values depending on the content of the amino functional monomer. One example of the copolymer is dimethylaminoethyl methacrylate, which is soluble under basic conditions.

Another example of the removal characteristic that the coatings 36A, 36B, 36C may exhibit is differential thermal release in aqueous conditions. In these examples, different lower critical solution temperature (LCST) polymers, upper critical solution temperature (UCST) polymers, and/or waxes with different melting temperatures may be selected for the coatings 36A, 36B, 36C. LCST polymers, such as poly(N-isopropyl acrylamide), become soluble in water when exposed to a temperature that is lower than a critical temperature; and conversely, UCST polymers, such as poly(N-acryloyl glycinamide) and poly(acrylamide-co-acrylonitrile), become soluble in water when exposed to a temperature above a critical temperature (which may be unique to each polymer). Examples of suitable waxes include beeswax (Tₘ from about 62° C. to about 65° C.), paraffin wax (Tₘ from about 46° C. to about 61° C.), and different molecular weight petroleum waxes (Tₘ from about 37° C. to about 95° C., where the Tₘ increases with the number of carbon atoms).

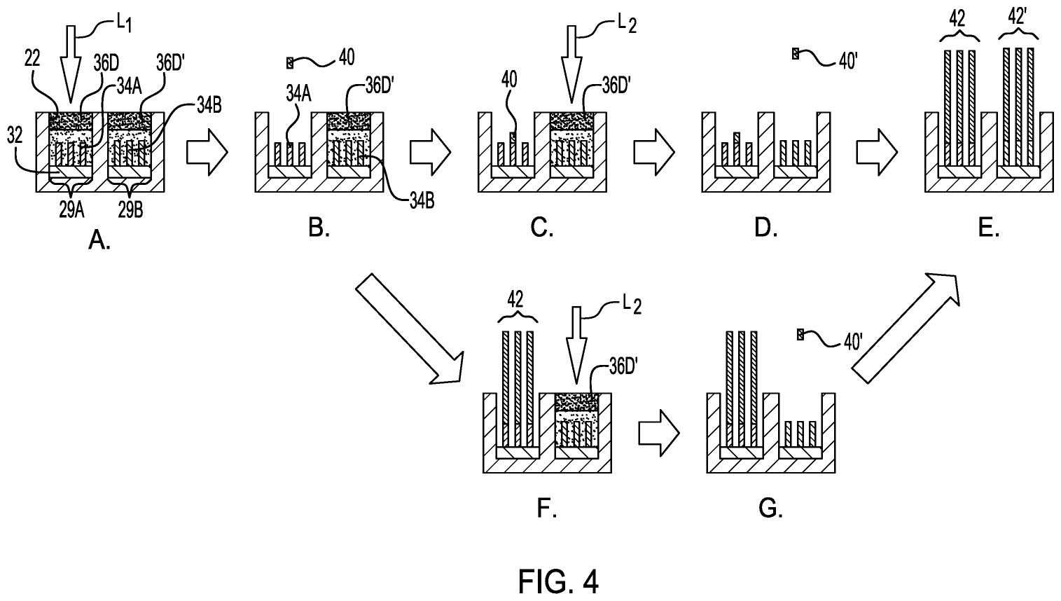

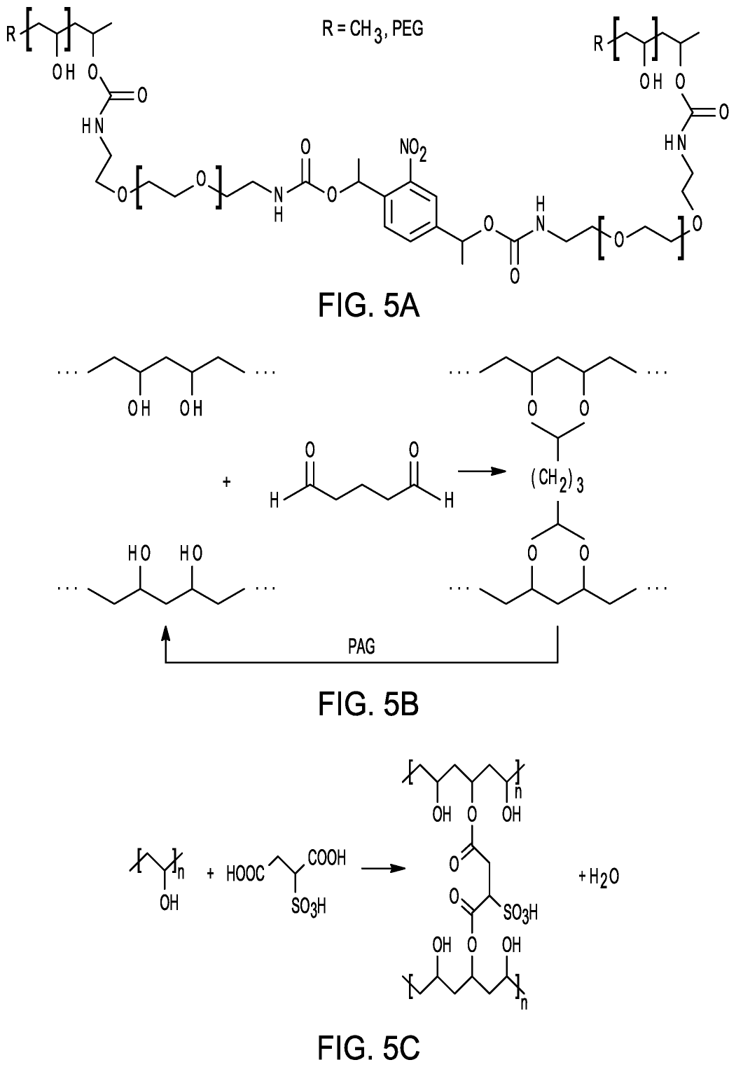

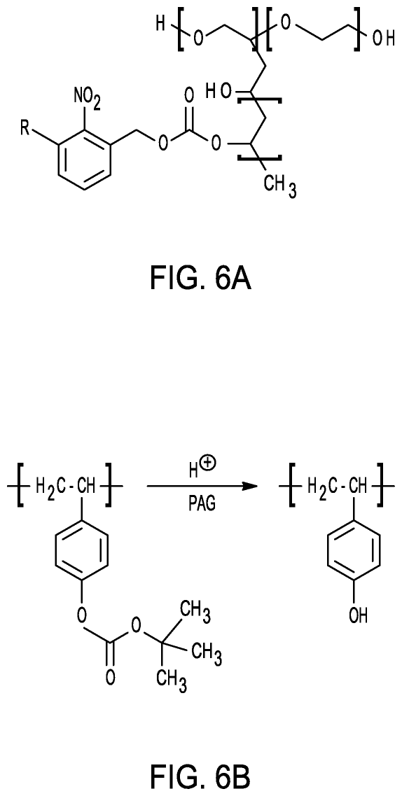

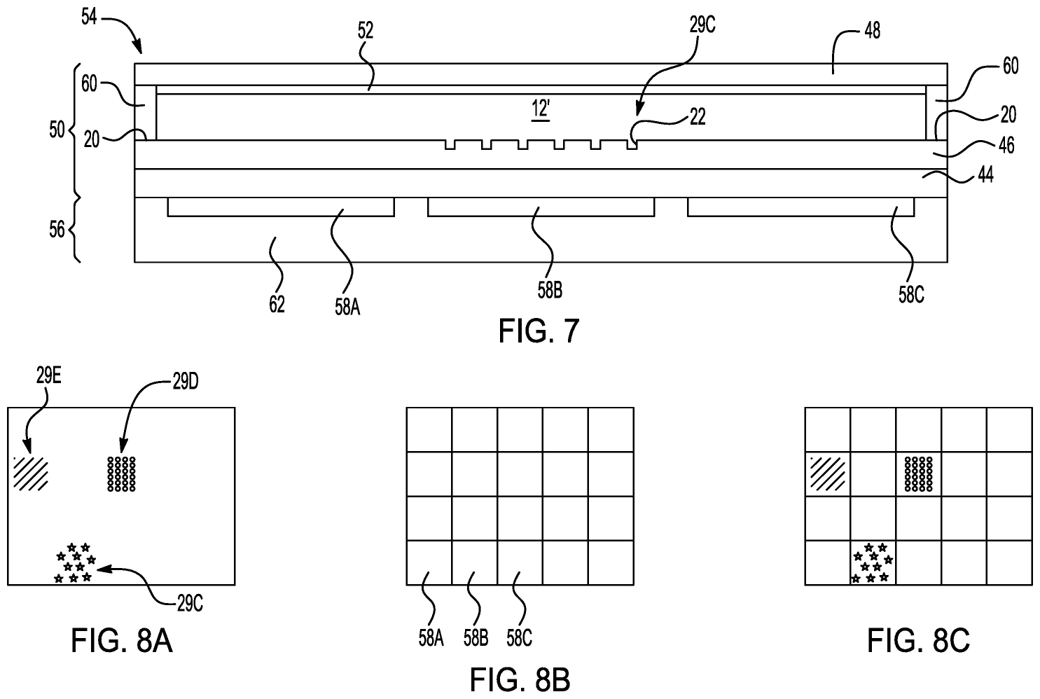

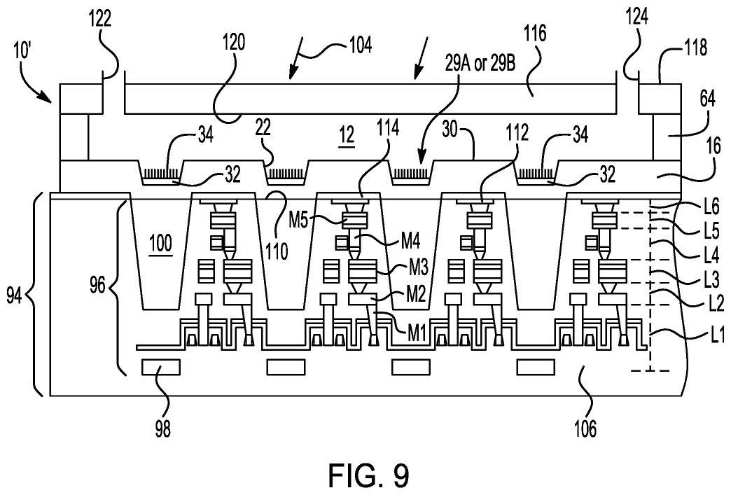

In other examples, one or more removable coatings 36D or 36D′ (see FIG. 4 at A., B., C., and F.) coat(s) the reactive regions 29A, 29B, and the removal characteristic of these coatings 36D, 36D′ is photo-reactivity. The coatings 36D and 36D′ may be the same and thus responsive to the same wavelength(s), or the coatings 36D, 36D′ may include materials that are responsive to different ranges of light wavelengths (e.g., L₁ and L₂, shown at FIG. 4 at A. and C.). Further, while not shown in FIG. 4, additional coatings, each having individual light removal characteristics, may be used in examples where additional selectivity is desired. The photo-reactive protective layers 36D, 36D′ may be positioned in each of the depressions 22 or over each of the protrusions 24 in a similar manner to the coatings 36A, 36B, 36C shown in FIG. 2A and FIG. 3A. As described herein, with photo-reactive coatings, the removable mechanism may be the light itself, or the heat produced by the light.

Alternatively, while not shown in FIG. 4, either of the coatings 36D or 36D′ may be in the form of a single layer that extends over all of the reactive regions 29A, 29B (including over the interstitial regions 30). In such examples, the other coating 36D′ or 36D is not included. A single photo-reactive protective layer 36D or 36D′ may be used in these examples because the photo-reactivity renders the layer 36D or 36D′ selectively removable via light exposure. Light enables both spatial and temporal control. Thus, a certain portion of the photo-reactive protective layer 36D or 36D′ (e.g., that overlies a specific reactive region 29A, 29B or a specific subset of the reactive regions 29A, 29B that is/are to be involved in subsequent reaction(s)) may be removed by exposing the certain portion to light.

In an example, the photo-reactive protective layer 36D or 36D′ is a hydrophilic polymer cross-linked with a photo-cleavable cross-linker. In this example, a high degree of cross-linking within the polymer renders the coating 36D, 36D′ structurally stable, thus minimizing release or exposure of the reactive entity 34A or 34B (underlying the photo-reactive protective layer 36D, 36D′) to an external environment. Upon exposure to a certain wavelength, the photo-cleavable cross-linker is cleaved, which produces a chemical structure that is spatially degraded and soluble. As such, controlled light exposure enables the layer 36D, 36D′ to be removed (e.g., to be washed away) using a suitable aqueous solvent, such as water.

Examples of hydrophilic polymers that may be used in these examples of the layer 36D, 36D′ include PAZAM co-polymerized with or substituted with a photo-cleavable group such as N-hydroxyalkyl acrylamide, polyethylene glycol (PEG), poly(hydroxyethyl acrylate), hydroxypropyl methylcellulose, poly(vinyl pyrrolidone), and polyvinyl alcohol (PVA). Examples of photo-cleavable cross-linkers for these examples of the layers 36D, 36D′ include o-nitrobenzyl moieties and coumarin moieties.

To enable cross-linking by curing, the hydrophilic polymers described herein may be chemically modified with reactive groups, such as acrylate or a reactive ester. Radical polymerization (via acrylate or methacrylate groups) may be used to cross-link the hydrophilic polymer and the photo-cleavable cross-linker, or cross-linking can be performed via other methods of chemical curing, such as thiolene coupling, amine-reactive ester (via p-nitrophenyl ester or N-hydroxy-succinimide ester) coupling, or amine-epoxy coupling.

In one specific example, the hydrophilic polymer is a polyvinyl alcohol/polyethylene glycol graft copolymer, and the photo-cleavable cross-linker is a coumarin or o-nitrobenzyl moiety. The coumarin or o-nitrobenzyl moieties may covalently conjugate with poly(ethylene glycol) of the polyvinyl alcohol/polyethylene glycol graft copolymer. An example is depicted in FIG. 5A. The coumarin or o-nitrobenzyl is cleavable upon exposure to ultraviolet (UV) (100 nm to 400 nm) or blue light (450 nm to 495 nm), which leads to decreased cross-linking density of the polymer coating. Thus, upon light exposure, the cleaved polymer coating becomes hydrophilic and washable by an aqueous solution.

In another example, the photo-reactive protective layer 36D or 36D′ is a hydrophilic polymer cross-linked with an acid-labile cross-linker. With the acid-labile cross-linker, an acid species can be generated in the presence of a photoacid generator (PAG) and upon exposure to UV/visible light, which changes the pH and cleaves the cross-linker, thus rendering the polymeric coating 36D or 36D′ more soluble (and easily removable via rinsing). This enables the layer 36D, 36D′ to be washed away, e.g., using an aqueous solvent.

Any suitable hydrophilic polymer described herein may be cross-linked with the acid-labile cross-linker. Examples of acid-labile cross-linkers include aldehyde cross-linkers, carboxylic acid cross-linkers, terephthalic acid cross-linkers, suberic acid cross-linkers, sulfosuccinic acid cross-linkers, glutaraldehyde cross-linkers, or glyoxal cross-linkers.

In a specific example, the hydrophilic polymer is a polyvinyl alcohol/polyethylene glycol graft copolymer, and the acid-labile cross-linker includes acetal groups.

An additional example of the hydrophilic polymer cross-linked with the acid-labile crosslinker is shown in FIG. 5B. This example depicts the cross-linking of polyvinyl alcohol (PVA) using glutaraldehyde (GA) via acid-labile acetal groups. Yet another example is shown in FIG. 5C. This example depicts the cross-linking of PVA using sulfosuccinic acid via ester groups.

As mentioned, the photoacid generator that is used may be reactive to UV light or visible light. One example of a suitable photoacid generator (that is reactive to UV light) is a sulfonium-based photoacid generator. An example of a suitable photoacid generator (that is reactive to visible light) is an arylazo sulfone-based photoacid generator.

In yet another example, the photo-reactive protective layer 36D or 36D′ is a polymer capped with a photo-cleavable hydrophobic group or an acid-labile group. In this example, cross-linking is not utilized. The photo-cleavable hydrophobic group or acid-labile protective groups on the polymer side chains render the polymer more hydrophobic. These examples of the photo-reactive protective layer 36D or 36D′ undergo a phase transition from hydrophobic to hydrophilic upon exposure to light. In an example, the polymer is polymethacrylic acid, polyphenol, polyvinyl alcohol, or polyvinyl alcohol/polyethylene glycol graft copolymer, which is functionalized with the photo-cleavable hydrophobic group, such as a coumarin or o-nitrobenzyl moiety. These photo-cleavable groups are generally hydrophobic, which prevents the photo-reactive protective layer 36D from being removed and washed away by an aqueous solution. Upon exposure to UV or visible light, these hydrophobic groups will be cleaved, returning the polymer coating back to its hydrophilic form. A specific example is shown in FIG. 6A. The cleavage of the o-nitrobenzyl group shown in FIG. 6A renders the coating 36D, 36D′ more hydrophilic, which enables the coating to be removed/washed using an aqueous solution. In another example, the hydrophilic polymer is polymethacrylic acid, polyphenol, polyvinyl alcohol, or polyvinyl alcohol/polyethylene glycol graft copolymer, which is functionalized with an acid-labile group. A specific example is shown in FIG. 6B. An acid species can be generated in the presence of a photoacid generator (PAG) and upon exposure to UV/visible light, the acid-labile group is cleaved. In the example shown in FIG. 6B, the cleavage of tert-butyl carbonate groups leads to the hydrophilic polyphenol coating, which is washable and removable by an aqueous solution.

In yet another example, at least one of the independently removable photo-reactive coatings 36D or 36D′ (positioned over the reactive regions 29A, 29B) is a composite that includes a thermo-responsive polymer and a photo-thermal additive (or photo-thermal filler). In this example, a respective one of the removable coating(s) 36D or 36D′ may be positioned in each of the depressions 22 or over each of the protrusions 24 in a similar manner to the coatings 36A, 36B, 36C shown in FIG. 2A and FIG. 3A, or one removable coating 36D or 36D′ may be a single layer that extends over all of the reactive regions 29A, 29B (including over the interstitial regions 30).

The thermo-responsive polymer used in the composite that forms the protective layer 36D or 36D′ may be a suitable polymeric material that undergoes a phase transition upon exposure to a predetermined change in temperature. As an example, the thermo-responsive polymer may be a polymer that melts upon reaching a certain temperature. As another example, the thermo-responsive polymer may be a polymer that transitions from a hydrophobic state to a hydrophilic state upon reaching a certain temperature, e.g., UCST (upper critical solution temperature) polymers, such as poly(acrylamide-co-acrylonitrile). As yet another example, the thermo-responsive polymer may be a polymer that transitions from a hydrophilic state to a hydrophobic state upon reaching a certain temperature, e.g., LCST (lower critical solution temperature) polymers, such as poly(N-isopropylacrylamide) or PNIPAAm.

As such, the thermo-responsive polymer may be selected from the group consisting of polylactic acid, poly(lactic-co-glycolic) acid, polycaprolactone, agarose, wax, poly(acrylamide-co-acrylonitrile), poly(N-isopropylacrylamide), cyclodextrins, polyethylene glycol homopolymer, polyethylene glycol graft copolymer, polyethylene block copolymer, and a combination thereof.

The photo-thermal additive or photo-thermal filler included in the composite of the protective layer 36D or 36D′ may be nano-sized or any micro-sized structures that absorb light (e.g., photonic) energy at a certain wavelength range and convert the absorbed energy to heat. In an example, the structures are spherical nanoparticles or microparticles. In another example, the structures are non-spherical nanoparticles or microparticles, such as cubes, triangular prisms, rod shaped, platelets, cage-like (e.g., non-spherical, hollow particles having a porous shell), tubes, etc. In still another example, the structures are irregularly shaped nanoparticles or microparticles. The dimensions of the structures may vary depending upon their shape. In the examples disclosed herein, the largest dimension (e.g., diameter, length, median, etc.) of the structure may be on the nanoscale, and thus ranges from about 1 nm to less than 1000 nm, or on the microscale, and thus ranges from about 1 μm to less than 1000 μm. In some examples, the structures are nanoparticles having a diameter of greater than or equal to 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, or greater than or equal to 100 nm.

The absorption and conversion is due, in part, to the structures' plasmonic properties (e.g., gold particles, silver particles, polypyrrole, graphene, and the like). Alternatively, the photo-thermal filler may be a photo-isomerizable dopant with a heat activation barrier, such as azobenzene. In an example, the photo-thermal filler (or photo-thermal additive) may be selected from the group consisting of gold nanoparticles, silver nanoparticles, iron oxide nanoparticles, polypyrrole particles, graphene sheets, carbon nanotubes, carbon nanodots, black phosphorus particles, azobenzene particles, and combinations thereof. It is to be understood that the morphology of the photo-thermal additive(s)/filler(s) may be tuned or otherwise manipulated according to the desired wavelength that is to be used for removing the coating 36D, 36D′. For instance, gold nanostructures having a higher aspect ratio (e.g., gold nanorods) absorb higher wavelengths of light (e.g., red-to-NIR light) relative to gold nanospheres (e.g., which absorb blue light).

The photo-thermal additive(s)/filler(s), in combination with the thermo-responsive polymer(s), may be distributed in a polymer matrix (i.e., composite) that forms the removable coating 36D or 36D′. Upon exposure to UV or visible light, energy absorbed by the photo-thermal additive(s) is converted to heat, which induces a phase transition in the polymer matrix (due to the presence of the thermo-responsive polymer). This phase transition causes the composite-based photo-reactive coating 36D or 36D′ to soften or dissolve. The softened or dissolved coating 36D or 36D′ can then be removed using an aqueous solvent.

As described, in some instances, two different photo-reactive protective layers 36D, 36D′ are utilized. In these examples, the layers 36D and 36D′ are responsive (cleaved) at different ranges of light wavelengths L₁ and L₂, and the layers 36D and 36D′ may be any of the light-reactive coating materials described herein, provided that the coatings 36D and 36D′ are responsive to the different ranges of light wavelengths L₁, L₂. As an example, one of the layers 36D, 36D′ may be responsive to light wavelengths L₁ ranging from 220 nm to 370 nm, while the other layer 36D′, 36D may be responsive to light wavelengths L₂ ranging from 440 nm to 800 nm.

Any of the example coatings (e.g., 36A, 36B, 36C, 36D, or 36D′) set forth herein may be used in combination in order to obtain multi-layer coatings. Examples of some multi-layer removable coatings 38A, 38B are shown in FIG. 2B and FIG. 3B. In this example, at least one of the plurality of independently removable coatings includes a plurality of sub-layers (i.e., is a multi-layer removable coating 38A, 38B); and the plurality of sub-layers defines the removal characteristic of the at least one of the plurality of independently removable coatings. When multiple light triggered removable materials are included in the multi-layer removable coatings 38A, 38B, the wavelengths should be selected to avoid overlap and to prevent premature removal of a particular coating.

The sub-layers of the multi-layer removable coatings 38A, 38B may be any of the materials set forth herein for the coatings 36A, 36B, 36C, 36D, and 36D′. The incorporation of the coatings 36A, 36B, 36C, 36D, and/or 36D′ into a stack enables the same coatings 36A, 36B, 36C, 36D, and/or 36D′ to be used in different orders, which imparts different removal characteristics to the overall multi-layer removable coatings 38A, 38B. The overall removal characteristics of each multi-layer removable coating 38A, 38B will depend upon the coatings 36A, 36B, 36C, 36D, and 36D′, and the order of the coatings 36A, 36B, 36C, 36D, and 36D′in the particular stack.

In the examples shown in FIG. 2B and FIG. 3B, the multi-layer removable coatings 38A, 38B require sequential treatments in order to reveal an underlying reactive region 29A, 29B. The sequential treatments will depend upon the sub-layers, e.g., coatings 36A, 36B, 36C, 36D, and/or 36D′ that are included in the stack. The sub-layers in each of the multi-layer removable coatings 38A, 38B may be selected so that some of the reactive regions 29A, 29B remain coated with at least some sub-layers of the multi-layer removable coating 38A or 38B, even when other sub-layers of the multi-layer removable coating 38A or 38B are removed or when another multi-layer removable coating 38B or 38A is completely removed.

In the example show in FIG. 2B and FIG. 3B, the removal of the multi-layer coating 38A involves the sequential dissolution of coating 36B and then coating 36A, while the removal of the multi-layer coating 38B involves the sequential dissolution of coatings 36C, 36A, and then 36B.

Per Table 2, in some examples, the coatings 36A and 36C may be selected so that they are not soluble in the same solvent (or are not susceptible to the same removal characteristic). In these examples, the removal of the single layer coating 36A will not affect either of the multi-layer coatings 38A, 38B. In these examples, the multi-layer coatings 38A, 38B may then be removed in any order that is desirable for exposure of the respective underlying reactive regions 29A, 29B. For example, the multi-layer coating 38A may be dissolved before the multi-layer coating 38B, or the multi-layer coating 38B may be dissolved before the multi-layer coating 38A.

Also per Table 2, in other examples where solubility-based removable coatings 36A, 36B, and/or 36C are utilized, the coatings 36A and 36C may be selected so that they are soluble in the same solvent (or are susceptible to the same removal characteristic). In some of these examples, the removal of the single layer coating 36A will also remove the first two coatings (sub-layers) 36C, 36A of the multi-layer coating 38B as long as the solvent exposure time is long enough for such removal. In these particular examples, the removal of the coatings (sub-layers) 36C, 36A of the multi-layer coating 38B will expose coating 36B. The coatings (sub-layers) 36B of each of the multi-layer coatings 38A, 38B may then be removed simultaneously. As such, in these particular examples, the multi-layer coating 38B may be removed before the multi-layer coating 38A. Alternatively, the removal of the single layer coating 36A may be performed so that the first coating (sub-layer) 36C of the multi-layer coating 38B is removed but so that the second coating (sub-layer) 36A of the multi-layer coating 38B remains intact. In these particular examples, the multi-layer coating 38A or the remainder of the multi-layer coating 38B (i.e., sub-layers/coatings 36A, 36B) may then be removed in any order that is desirable for exposure of the respective underlying reactive regions 29A, 29B. For example, the multi-layer coating 38A may be dissolved before the remaining sub-layers of the multi-layer coating 38B, or the remaining sub-layers of the multi-layer coating 38B may be dissolved before the multi-layer coating 38A.

With the multi-layer coatings 38A, 38B, the sub-layers may be selected to be barriers to reagents (e.g., sequencing reagents, tagmentation buffers, etc.) that are used in the reactions with the exposed reactive regions 29A, 29B. As barriers to reaction reagents, the sub-layers will minimize or prevent these reagents from penetrating into the remaining sub-layers.

Any of the methods disclosed herein may be used to generate the multi-layer coatings 38A, 38B. For example, in a roll-to-roll coating system, multiple sequential coating heads (each coating a unique set of rectangular patches or stripes) can build up different layers (of the multi-layer coatings 38A or 38B) on discrete regions of a substrate (as shown in FIG. 2B and FIG. 2C, noting, however, that each coating layer does not have to be deposited in each pass). In a wafer coating system, inkjet, screen, stencil, or precision dispense coating may be used to build up layers (of the multi-layer coatings 38A or 38B), with each material printing a unique 2D coating region.

Curing steps may be performed after each sub-layer is deposited depending on the material used.

Referring now to FIG. 2C and FIG. 3C through FIG. 3E, still other examples of the independently removable coatings 36, 36′, 36″, 36″′ are depicted. In these examples, the coating 36 is, or the coatings 36′, 36″, 36″′ are formed of the same material (and may be any of the materials set forth herein for the coatings 36A, 36B, 36C, 36D, 36D′) but the coating 36 has a variable thickness or each of the plurality of coatings 36′, 36″, 36″′ has a different thickness. As such, while the coating 36 is, or the coatings 36′, 36″, 36″′ are susceptible to the same removal characteristic, the rates at which the coating 36 is removed (e.g., via dissolution, light exposure, etc.), or at which the coatings 36′, 36″, 36″′ are removed, is/are different due to the different thicknesses T₁, T₂, T₃.

In the example shown in FIG. 2C, each of the depressions 22 has a removable coating 36′, 36″, 36″′ with a different thickness positioned over the reactive region 29A, 29B associated with the depressions 22. In one example, the thickness T₁<the thickness T₂<the thickness T₃, and T₁ ranges from about 10 nm to about 500 nm, T₂ ranges from about 100 nm to about 1 μm, and T₃ ranges from about 500 nm to about 10 μm. It is to be understood that in an array of depressions 22, each depression 22 can have a removable coating 36′, 36″, 36″′ with a different thickness, or subsets of depressions 22 in the array can have coatings 36′, 36″, 36″′ with different thicknesses. Each subset may include two or more depressions 22.

In the example shown in FIG. 3C, each of the protrusions 24 has a removable coating 36′, 36″, 36″′ with a different thickness positioned over the reactive region 29A, 29B associated with the protrusions 24. Like FIG. 2C, the thickness T₁<the thickness T₂<the thickness T₃, and T₁ ranges from about 10 nm to about 500 nm, T₂ ranges from about 100 nm to about 1 μm, and T₃ ranges from about 500 nm to about 10 μm. It is to be understood that in an array of protrusions 24, each protrusion 24 can have a removable coating 36′, 36″, 36″′ with a different thickness, or subsets of protrusions 24 in the array can have coatings 36′, 36″, 36″′ with different thicknesses. Each subset may include two or more protrusions 24.

In the examples shown in FIG. 3D and FIG. 3E, a single removable coating 36 is positioned over each of the protrusions 24 (and reactive region 29A, 29B), but the removable coating 36 has a variable thickness. As such, the thickness T₁, T₂, or T₃, of the removable coating 36 is thinner over some of the protrusions 24 (and reactive region 29A, 29B), and is thicker over some other of the protrusions 24 (and reactive region 29A, 29B). In the example shown in FIG. 3D, the removable coating 36 has a step-wise gradient that increases from T₁ to T₂ to T₃ across, e.g., the width, of the patterned structure 14B. In the example shown in FIG. 3E, the removable coating 36 has a substantially linear gradient that increases from T₁ to T₂ to T₃ across, e.g., the width, of the patterned structure 14B. In both examples, each protrusion 24 or subset of protrusions 24 is positioned beneath a different thickness T₁, T₂, or T₃, of the removable coating 36.

While three different thicknesses are shown in FIG. 2C, FIG. 3C, FIG. 3D, and FIG. 3E, it is to be understood that any number of different coating thicknesses may be used (e.g., 2 or greater than 3). The number of different coating thicknesses will depend upon the number of depressions 22 or protrusions 24 in a channel 12, and the number of subsets (if any) of depressions 22 or protrusions 24. The number of subsets, in turn, is based on the desired number of reactions that are to be performed within a single flow cell channel 12.

### Methods for Using the Flow Cells

Examples of the flow cells 10 described herein may be used in analysis methods, including nucleic acid sequencing. The independently removable coatings 36 described herein (36A, 36B, 36C, 36D, 36D′, 36′, 36″, or 36″′) enable select reactive regions 29A, 29B to be uncovered and rendered ready for analysis/chemical reaction, while also enabling other reactive regions 29A, 29B to remain covered so that they do not participate in the then-current analysis/chemical reaction.

An example of the method for using some examples of the flow cell 10 includes i) selectively removing at least one of a plurality of independently removable coatings 36A, 36B, 36C, 36D, 36D′, or 36′, 36″, 36″′ respectively positioned over each of a plurality of reactive regions 29A, 29B spatially separated from one another across a substrate 16 or 18, thereby exposing at least one of the plurality of reactive regions 29A, 29B and a reactive entity 34 at the at least one of the plurality of reactive regions 29A, 29B, wherein each of the plurality of reactive regions 29A, 29B includes a polymeric hydrogel layer 32 and the reactive entity 34 attached to the polymeric hydrogel layer 32, and ii) initiating a reaction involving the reactive entity 34. In some examples of the method, at least one of the independently removable coatings 36 is the composite including the thermo-responsive polymer and the photo-thermal additive (and thus at least one of the removable coatings 36 is the photo-reactive coating 36D or 36D′).

Another example of the method for using other examples of the flow cell 10 includes i) selectively removing an independently removable portion of a coating 36 positioned over each of a plurality of reactive regions 29A, 29B spatially separated from one another across a substrate 16 or 18, thereby exposing at least one of the plurality of reactive regions 29A, 29B and a reactive entity 34 at the at least one of the plurality of reactive regions 29A, 29B, whereby at least one other independently removable portions remains unaffected; and ii) initiating a reaction involving the reactive entity 34.

Some example methods utilize the independently removable coatings 36A, 36B, 36C, where the removal characteristic of each coating is solubility. In these examples, selectively removing the at least one of the plurality of independently removable coatings 36A, 36B, 36C involves exposing the plurality of independently removable coatings 36A, 36B, 36C to a predetermined solvent that dissolves the at least one of the plurality of independently removable coatings 36A, 36B, 36C and does not dissolve the at least one other of the independently removable coatings. The solvent or aqueous solution that is used will depend upon the respective materials for the coatings 36A, 36B, 36C. In some examples of this method, the at least one of the plurality of independently removable coatings includes a plurality of sub-layers (i.e., is the multi-layer coating 38A or 38B); and selectively removing the at least one of the plurality of independently removable coatings involves exposing the at least one of the plurality of independently removable coatings to a sequential removal treatment that sequentially dissolves each of the plurality of sub-layers. The combination of solvents or aqueous solutions that is used will depend upon the respective materials of the sub-layers in the multi-layer coating 38A or 38B.

In examples where each of the plurality of removable coatings 36′, 36″, 36″′ has a different thickness T₁, T₂, T₃; selectively removing the at least one of the plurality of independently removable coatings 36′, 36″, 36″′ involves exposing the plurality of independently removable coatings 36′, 36″, 36″′ to a solvent for a predetermined time. The predetermined time is selected so that the thinnest independently removable coating 36′ or 36″ that is present on the flow cell 10 will be removed, and any thicker independently removable coating(s) 36″ and/or 36″′ will remain partially intact. It is to be understood that during solvent exposure, some of the thicker independently removable coating(s) 36″ and/or 36″′ will be removed (because the coatings 36′, 36″, 36″′ are formed of the same material), but the presence of additional material in the thicker coating(s) 36″ and/or 36″′ prevents them from being completely removed. At least one of the thicker independently removable coating(s) 36″ and/or 36″′ remains partially intact after the then-currently targeted coating 36 or 36″ is removed and solvent exposure ceases as long as a thicker coating (e.g., 36″ and/or 36″′) than the coating (e.g., 36′ or 36″) being targeted for removal is present. In these examples, the solvent exposure time will depend upon the dissolution rate of the material of the coatings 36′, 36″, 36″′.

In still other examples, selectively removing the independently removable portion of the coating 36 involves exposing the coating 36 to a predetermined solvent for a predetermined time so that the independently removable portion is removed and a remainder of the coating 36 is left intact. This method may be used with the examples shown in FIG. 3D and FIG. 3E. In these examples, the solvent exposure time will depend upon the dissolution rate of the material of the coating 36. At the selected exposure time, the thinnest portion of the coating 36 (e.g., with thickness T₁ or T₂) that is present on the flow cell 10 will be removed, and any thicker portions (e.g., with thickness T₂ or T₃) remain partially intact. It is to be understood that during solvent exposure, some of the thicker portions (e.g., with thickness T₂ or T₃) will be removed, but the presence of additional material in the thicker portions (e.g., with thickness T₂ or T₃) prevents these portion(s) from being completely removed. As long as a thicker portion of the coating 36 is present on the flow cell 10 when the targeted (thinner) portion is removed, this thicker portion will remain partially intact after the then-currently targeted portion of the coating 36 is removed and solvent exposure ceases.

After selective removal of the individually removable coating 36A, 36B, 36C, 36′, 36″, or 36″′, a reaction may be performed at the exposed reactive region(s) 29A, 29B. The reaction(s) performed will depend upon the reactive entity 34A, 34B at the reactive region 29A, 29B. In one example, the reactive entity 34A, 34B is an immobilized transposome complex and the reaction involves tagmentation of an introduced DNA sample. In another example, the reactive entity 34A, 34B is the primer set and the reaction involves seeding of a library template strand, amplification of the library template strand to form amplicons, and then sequencing of the amplicons.

Other examples of the method involve utilizing/removing the photo-reactive protective layer 36D or 36D′. In some of these examples, the substrate 16, 18 includes a plurality of depressions 22; each of the plurality of reactive regions 29A or 29B is positioned within a respective one of the plurality of depressions 22; and each of the independently removable coatings 36D or 36D′ covers a respective one of the plurality of reactive regions 29A or 29B (similar to FIG. 1B). In other of these examples, the substrate 16, 18 includes a plurality of protrusions 24, each of the plurality of reactive regions 29A or 29B is positioned at a respective one of the plurality of protrusions 24, and each of the independently removable coatings 36D or 36D′ covers a respective one of the plurality of protrusions 24 (similar to FIG. 1C).

Selectively removing the at least one of the plurality of independently removable coatings 36D or 36D′ may involve exposing the at least one of the plurality of independently removable coatings 36D or 36D′ to light without exposing the at least one other of the independently removable coatings 36D′ or 36D to the light, thereby rendering the at least one of the plurality of independently removable coatings 36D or 36D′ removable using an aqueous solvent.

Two examples of this method are shown in FIG. 4, at A. through E. and at A., B., F., G., and E. In each of these methods, the photo-reactive protective layer 36D is shown as being applied over the reactive region 29A in one of the depressions 22, and a different photo-reactive protective layer 36D′ is shown as being applied over the reactive region 29B in a different depression 22. In this particular example, the reactive entity 34A in one of the depressions 22 is different than the reactive entity 34B in the other of the depressions 22. As one example, the reactive entities 34A, 34B may be two different primer sets. It is to be understood, however, that in some examples, the same photo-reactive protective layer 36D or 36D′ may be applied over each of reactive regions 29A and 29B, and the same or different reactive entity 34A or 34B may be applied in two adjacent depressions 22.

At A., light (within the first range of light wavelengths L₁, represented by the arrow at A.) is used to selectively remove the photo-reactive protective layer 36D that overlies the reactive entity 34A. It is to be understood that the range of light wavelengths L₁ that is used to remove the photo-reactive protective layer 36D will depend, in part, upon the material(s) used in the photo-reactive protective layer 36D. For example, when the layer 36D includes a material that absorbs blue light to near-infrared light, L₁ may represent a range of 500 nm to 1200 nm. As a specific example, when the layer 36D includes spherical gold nanoparticles (e.g., when the layer 36D is the composite described herein), L₁ may represent a range of 500 nm to 600 nm. As another specific example, when the layer 36D includes gold nanorods, L₁ may represent a range of 600 nm to 1200 nm. As yet another specific example, when the layer 36D includes carbon nanodots, L₁ may represent a range of 400 nm to 800 nm. As still another specific example, when the layer 36D includes silver nanoparticles, L₁ may represent a range of 400 nm to 550 nm.

At B., a template strand 40 of a first library of template strands is introduced and seeds to the reactive entity 34A.

When the method continues at C., the seeded template strand 40 will occupy a certain density of the reactive entity 34A. Once seeding takes place, and light (within the second range of light wavelengths L₂, represented by the arrow at C. and F.) is used to selectively remove the photo-reactive protective layer 36D′ that overlies the reactive entity 34B. The second range of light wavelengths L₂ will depend, in part, upon the material used in the layer 36D′. For example, when the layer 36D′ includes a material that absorbs blue light to near-infrared light, L₂ may represent a range of 500 nm to 1200 nm. As a specific example, when the layer 36D′ includes spherical gold nanoparticles (e.g., when the layer 36D′ is the composite described herein), L₂ may represent a range of 500 nm to 600 nm. As another specific example, when the layer 36D′ includes gold nanorods, L₂ may represent a range of 600 nm to 1200 nm. As yet another specific example, when the layer 36D′ includes carbon nanodots, L₂ may represent a range of 400 nm to 800 nm. As still another specific example, when the layer 36D′ includes silver nanoparticles, L₂ may represent a range of 400 nm to 550 nm.

At D., another template strand 40′ of a second library of template strands is introduced and seeds to the reactive entity 34B. In an example, the template strand 40′ does not include an adapter for hybridizing to the reactive entity 34A, and thus will not seed in the depression(s) 22 containing the reactive entity 34A. In this example, different depressions 22 may individually contain different reactive entities 34A, 34B (e.g., different primer sets). In another example, the depressions 22 that are exposed during the seeding of the template strands 40 may be smaller than the depressions 22 that are exposed during the seeding of the template strands 40′. In these instances, the template strands 40′ may be sterically blocked from seeding in the depressions 22 containing the template strands 40.

When the same type of coating, e.g., 36D, is used with each reactive region 29A, 29B, the same light wavelengths are used to render the coatings removable, and the selectivity of the removal is controlled by the light exposure (i.e., where light is directed). Alternatively, when two different types of coatings 36D, 36D′ are used to cover respective reactive regions 29A, 29B, different light wavelengths within separate (and non-overlapping) ranges L₁, L₂ are used to remove the coatings 36D, 36D′, and the selectivity of the removal is controlled by the responsiveness of the coatings 36D, 36D′ to the different light wavelengths.

Each of the seeded template strands 40, 40′ is then respectively amplified and clustered across the respective reactive entities 34A, 34B, as shown in E. This generates first library template amplicons 42 in at least one of the depressions 22 and second library template amplicons 42′ in at least one other of the depressions 22.

When the method continues at F. (from B.), the seeded template strand 40 is amplified and clustered across the respective reactive entities 34A to generate the first library template amplicons 42. Once the cluster of amplicons 42 is generated using the reactive entity 34A, light is used to selectively remove the photo-reactive protective layer 36D′ that overlies the reactive entity 34B (also shown at F.). The light is within the second range of light wavelengths L₂ when a different coating 36D′ is used, or is within the same range of light wavelengths L₁ when the same coating 36D is used.

At G., another template strand 40′ of a second library of template strands is introduced and seeds to the reactive entity 34B. The seeded template strand 40′ is then amplified and clustered across the reactive entity 34B, as shown in E. This generates the second library template amplicons 42′ in at least one other of the depressions 22.

In still another example when the substrate 16, 18 includes a plurality of protrusions 24 and each of the plurality of reactive regions 29B is formed with a respective one of the plurality of protrusions 22, and each of the independently removable coatings is the photo-reactive protective layer 36D or 36D′, selectively removing the at least one of the plurality of independently removable coatings 36D or 36D′ involves exposing the at least one of the plurality of independently removable coatings 36D or 36D′ to light without exposing the at least one other of the independently removable coatings 36D′ or 36D to the light, thereby rendering the at least one of the plurality of independently removable coatings 36D or 36D′ removable using an aqueous solvent.

In another example when the photo-reactive protective layer 36D is a single layer positioned over a plurality of reactive regions 29A or 29B, the method involves selectively removing at least one portion of the coating 36D by exposing the portion(s) of the coating 36D to light without exposing the other portion(s) of the coating 36D to the light, thereby rendering the portion(s) removable using an aqueous solvent.

While FIG. 4 depicts specific examples with regard to the type of reaction that is performed, it is to be understood that different reactions can be performed when different reactive entities 34 are utilized. As one example, tagmentation may be performed when the reactive entities 34 are transposome complexes.

### Digital Fluidics Cartridge

Another example device that enables controlled access to reactive regions 29C is the digital fluidics cartridge 50 shown in FIG. 7, which includes a laminate film 44; a resin 46 positioned on the laminate film 44, the resin 46 patterned with a plurality of spatially separated reactive regions 29C; a lid 48 attached to a bonding region 20 of the resin 46; a fluid channel 12′ defined between the lid 48 and the resin 46; and a ground electrode 52 positioned on a surface of the lid 48 that faces the fluid channel 12′.

The digital fluidics cartridge 50 may be used in a digital fluidics system 54 which includes a digital fluidics instrument 56. The digital fluidics instrument 56 includes a plurality individually addressable control electrodes 58A, 58B, 58C.

In the example shown in FIG. 7, the laminate film 44 may be poly(ethylene terephthalate) or another thin plastic material. The thickness of the laminate film 44 may range from about 30 μm to about 200 μm. The laminate film 44 can act as a support for the resin 46. The resin 46 may be any examples of the resin materials set forth herein (e.g., for layer 28), and the reaction area(s) include(s) depressions 22 containing the polymeric hydrogel 32 and an example of the reactive entity 34 attached to the polymeric hydrogel 32 (polymeric hydrogel and reactive entity not shown in FIG. 7).

The lid 48 may be any of the example materials set forth herein, and may be attached to the bonding region 20 through any of the spacer layer materials set forth herein. The spacer layer is represented at reference numeral 60.

The ground electrode 52 may be any conductive material that can transmit the light used in the reaction(s) that is/are to take place in the reactive regions 29C. As examples the ground electrode 52 may be poly(3,4-ethylenedioxythiophene) (PEDOT) or indium tin oxide (ITO).

The plurality of individually addressable control electrodes 58A, 58B, 58C may be defined in a non-conductive (i.e., not electrically conductive) substrate 62 of the instrument 56. Examples of non-conductive substrates 62 include glass, rubber, porcelain, ceramic, and some polymeric materials.

The plurality individually addressable control electrodes 58A, 58B, 58C in the instrument 56 enable the regioselective movement of droplets along the surface of the resin 46. As shown in FIG. 7, the depressions 22 (containing the reactive regions 29A) are positioned over portions of the laminate film 44 that overlie the individually addressable control electrodes 58A, 58B, 58C. This positioning generates regions of selective functionalization.

FIG. 8A through FIG. 8C respectively illustrate top views of the resin 46 of the cartridge 50 with A) reactive regions 29C, 29D, 29E defined in three specific areas, B) the individually addressable control electrode (e.g., including electrodes 58A, 58B, 58C, etc.) array of the instrument 56, and C) the cartridge 50 in position on the instrument 56. The electronic circuitry electrically connected to each of the electrodes 58A, 58B, 58C, etc. enables one or more of the electrodes 58A, 58B, 58C, etc. to be addressed at a particular time. Thus, electrically responsive fluids can be directed to one of the reactive regions 29C, 29D, 29E and not to the others depending on which of the electrodes 58A, 58B, 58C is addressed.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

What is claimed is:

1. A flow cell, comprising:
a substrate;
a plurality of reactive regions spatially separated from one another across the substrate, each of the plurality of reactive regions including:
a polymeric hydrogel layer; and
a reactive entity attached to the polymeric hydrogel layer; and
a plurality of independently removable coatings respectively positioned over each of the plurality of reactive regions;
wherein at least one of the plurality of independently removable coatings is a composite that includes a thermo-responsive polymer and a photo-thermal additive.

2. The flow cell as defined in claim 1, wherein the reactive entity in each of the plurality of reactive regions is a primer set.

3. The flow cell as defined in claim 2, wherein the primer set is the same in each of the plurality of reactive regions.

4. The flow cell as defined in claim 2, wherein the primer set of at least one of the plurality of reactive regions is different than the primer set of at least one other of the plurality of reactive regions.

5. The flow cell as defined in claim 1, wherein:
the substrate includes a plurality of depressions;
each of the plurality of reactive regions is positioned within a respective one of the plurality of depressions; and
each of the plurality of independently removable coatings covers a respective one of the plurality of reactive regions.

6. The flow cell as defined in claim 1, wherein:
the substrate includes a plurality of protrusions;
each of the plurality of reactive regions is positioned at a respective one of the plurality of protrusions; and
each of the plurality of independently removable coatings covers a respective one of the plurality of protrusions.

7. The flow cell as defined in claim 1, wherein the thermo-responsive polymer is selected from the group consisting of polylactic acid, poly(lactic-co-glycolic) acid, polycaprolactone, agarose, wax, poly(acrylamide-co-acrylonitrile), poly(N-isopropylacrylamide), cyclodextrin, polyethylene glycol homopolymer, polyethylene glycol graft copolymer, polyethylene block copolymer, and combinations thereof.

8. The flow cell as defined in claim 1, wherein the photo-thermal filler is selected from the group consisting of gold nanoparticles, silver nanoparticles, iron oxide nanoparticles, polypyrrole, graphene, carbon nanotubes, carbon nanodots, black phosphorus, azobenzene, and combinations thereof.

9. A method, comprising:
selectively removing at least one of a plurality of independently removable coatings respectively positioned over each of a plurality of reactive regions spatially separated from one another across a substrate, thereby exposing at least one of the plurality of reactive regions and a reactive entity at the at least one of the plurality of reactive regions;
wherein each of the plurality of reactive regions includes a polymeric hydrogel layer and the reactive entity attached to the polymeric hydrogel layer; and
wherein at least one of the plurality of independently removable coatings is a composite including a thermo-responsive polymer and photo-thermal additive; and
initiating a reaction involving the reactive entity.

10. The method as defined in claim 9, wherein selectively removing the at least one of the plurality of independently removable coatings involves:
exposing the at least one of the plurality of independently removable coatings to light, thereby rendering the at least one of the plurality of independently removable coatings that has been exposed to the light susceptible to removal using an aqueous solvent; and
removing the at least one of the plurality of independently removable coatings using the aqueous solvent.

11. The method as defined in claim 9, wherein:
the substrate includes a plurality of depressions;
each of the plurality of reactive regions is positioned within a respective one of the plurality of depressions; and
each of the plurality of independently removable coatings covers a respective one of the plurality of reactive regions.

12. The method as defined in claim 9, wherein:
the substrate includes a plurality of protrusions;
each of the plurality of reactive regions is positioned at a respective one of the plurality of protrusions; and
each of the plurality of independently removable coatings covers a respective one of the plurality of protrusions.

13. The method as defined in claim 9, wherein the thermo-responsive polymer is selected from the group consisting of polylactic acid, poly(lactic-co-glycolic) acid, polycaprolactone, agarose, wax, poly(acrylamide-co-acrylonitrile), poly(N-isopropylacrylamide), cyclodextrin, polyethylene glycol homopolymer, polyethylene glycol graft copolymer, polyethylene block copolymer, and combinations thereof.

14. The method as defined in claim 9, wherein the photo-thermal filler is selected from the group consisting of gold nanoparticles, silver nanoparticles, iron oxide nanoparticles, polypyrrole, graphene, carbon nanotubes, carbon nanodots, black phosphorus, azobenzene, and combinations thereof.

15. The method as defined in claim 9, wherein the reactive entity in each of the plurality of reactive regions is a primer set.

16. The method as defined in claim 15, wherein the primer set is the same in each of the plurality of reactive regions.

17. The method as defined in claim 15, wherein the primer set of at least one of the plurality of reactive regions is different than the primer set of at least one other of the plurality of reactive regions.
